(19) United States
(12) Patent Application Publication
(10) Pub. No.: US 2021/0348242 A1
(43) Pub. Date: Nov. 11, 2021

(54) METHODS FOR MEASURING THE TITER AND POTENCY OF VIRAL VECTOR PARTICLES

(71) Applicant: VOYAGER THERAPEUTICS, INC., Cambridge, MA (US)

(72) Inventors: Lauren M. Drouin, Cambridge, MA (US); Carlo Ciatto, Brookline, MA (US); Patrick Starremans, Belmont, MA (US); Joseph Mombeleur, Bridgewater, MA (US); Rachael Ahern, Reading, MA (US); Timothy Boyd, Somerville, MA (US); Eric D. Horowitz, Norwell, MA (US)

(21) Appl. No.: 17/282,673

(22) PCT Filed: Oct. 4, 2019
(86) PCT No.: PCT/US2019/054606

§ 371 Date: Apr. 2, 2021

Related U.S. Application Data
(60) Provisional application No. 62/741,463, filed on Oct. 4, 2018.
(60) Provisional application No. 62/839,041, filed on Apr. 26, 2019.

(51) Int. Cl. C12Q 1/70

(52) U.S. Cl. CPC C12Q 1/70

(57) ABSTRACT

The present disclosure relates generally to methods for measuring the titer and potency of viral vector particles, including methods which use qPCR, ddPCR, or a combination thereof to measure the titer of the viral vector particles.

### CROSS REFERENCE TO RELATED APPLICATIONS

This application claims the benefit of: U.S. Provisional Patent Application No. 62/741,463, filed Oct. 4, 2018, entitled METHODS FOR MEASURING THE POTENCY OF AADC VIRAL VECTORS; and U.S. Provisional Patent Application No. 62/839,041, filed Apr. 26, 2019, entitled METHODS FOR MEASURING THE TITER AND POTENCY OF A VIRAL VECTOR; the contents of which are each incorporated herein by reference in their entirety.

### FIELD OF THE DISCLOSURE

The present disclosure relates generally to methods for measuring the titer and potency of viral vector particles, including methods which use qPCR, ddPCR, or a combination thereof to measure the titer of the viral vector particles.

### BACKGROUND

AAVs have emerged as one of the most widely studied and utilized viral vectors for gene transfer to mammalian cells. See, e.g., Tratschin et al., Mol. Cell Biol., 5(11):3251-3260 (1985) and Grimm et al., Hum. Gene Ther., 10(15):2445-2450 (1999), the contents of which are herein incorporated by reference in their entirety. Adeno-associated viral (AAV) vectors are promising candidates for therapeutic gene delivery and have proven safe and efficacious in clinical trials. The design and production of improved AAV particles for this purpose is an active field of study.

With the advent of development in the AAV field, there remains a need for improved systems and methods for producing AAV vectors (such as AAV particles) and corresponding therapeutic formulations for storage and delivery of the AAV particles. These include improved methods and systems for measuring, analyzing, quantifying and qualifying AAV particles and AAV formulations.

### SUMMARY

The present disclosure presents methods and examples for the improved use of qPCR and ddPCR in the analysis and quantification of viral materials such as AAV particles.

The present disclosure presents methods for measuring the titer of AAV vector particles in a formulation. In certain embodiments, the method includes: providing a formulation which includes a collection of AAV vector particles; and determing the titer of the AAV vector particles in the formulation using ddPCR. In certain embodiments, the method includes: providing a formulation which includes a collection of AAV vector particles; and determing the titer of the AAV vector particles in the formulation using qPCR. In certain embodiments, the method includes: providing a first formulation which includes a first collection of AAV vector particles; determing the titer of the AAV vector particles in the first formulation using qPCR; providing a second formulation which includes a second collection of AAV vector particles; and determing the titer of the AAV vector particles in the second formulation using ddPCR.

The present disclosure presents methods for measuring the potency of AAV vector particles in a formulation. In certain embodiments, the method includes: providing a first formulation which includes a first collection of AAV vector particles, wherein the first collection of AAV vector particles includes a polyncleotide encoding a payload molecule; determing the titer of the AAV vector particles in the first formulation using qPCR, ddPCR or a combination thereof; and measuring the potency of the AAV vector particles from the first formulation. In certain embodiments, the potency of the AAV vector particles from the first formulation is measured by: determining a multiplicity of infection (MOI) for the first collection of AAV vector particles based on the titer of the AAV vector particles in the first formulation; transducing the AAV vector particles from the first formulation into a target cell using the MOI for the first collection of AAV vector particles, and under conditions in which the target cell will produce the payload molecule; and measuring the amount of payload molecule produced from the AAV vector particles, such that the potency of the AAV vector particle is measured.

In certain embodiments, the titer of the AAV vector particles in the first formulation is determined using qPCR. In certain embodiments, the titer of the AAV vector particles in the first formulation is determined using ddPCR.

In certain embodiments, the step of measuring the amount of payload molecule produced from the first collection of AAV vector particles includes: lysing the target cells and collecting the resulting cell lysate sample; adding a molecule of interest to the cell lysate sample, wherein the molecule of interest interacts with the payload molecule to produce a product molecule; and measuring the amount of product molecule produced in the cell lysate, such that the potency of the AAV vector particles from the first formulation is measured. In certain embodiments, the the amount of product molecule produced is measured using Ultra High-Pressure Liquid Chromatography (UHPLC).

In certain embodiments, the method includes comparing the potency of AAV vector particles in the first formulation to the potency of reference AAV vector particles in a viral vector reference standard. In certain embodiments, the method includes: providing a first formulation which includes a first collection of AAV vector particles, wherein the first collection of AAV vector particles includes a polyncleotide encoding a payload molecule; determing the titer of the AAV vector particles in the first formulation using qPCR, ddPCR or a combination thereof; and measuring the potency of the AAV vector particles from the first formulation. In certain embodiments, the potency of the AAV vector particles from the first formulation is measured by: determining a multiplicity of infection (MOI) for the first collection of AAV vector particles based on the titer of the AAV vector particles in the first formulation; transducing the AAV vector particles from the first formulation into a target cell using the MOI for the first collection of AAV vector particles, and under conditions in which the target cell will produce the payload molecule; measuring the amount of payload molecule produced from the AAV vector particles, such that the potency of the AAV vector particle is measured; and comparing the potency of AAV vector particles in the first formulation to the potency of reference AAV vector particles in a viral vector reference standard.

In certain embodiments, the potency of the reference AAV vector particles in the viral vector reference standard is measured according to the following steps: providing a reference formulation which includes a collection of reference AAV vector particles, wherein the collection of reference AAV vector particles include a polyncleotide encoding the payload molecule; determing the titer of the reference AAV vector particles in the reference formulation using qPCR, ddPCR or a combination thereof; and measuring the potency of the reference AAV vector particles from the reference formulation. In certain embodiments, the potency of the reference AAV vector particles from the reference formulation is measured by: determining a multiplicity of infection (MOI) for the reference collection of AAV vector particles based on the titer of the reference AAV vector particles in the reference formulation; transducing the reference AAV vector particles from the reference formulation into a target cell using the MOI for the reference collection of AAV vector particles, and under conditions in which the target cell will produce the payload molecule; and measuring the amount of payload molecule produced from the reference AAV vector particles, such that the potency of the reference AAV vector particle is measured.

In certain embodiments, the titer of the reference AAV vector particles in the reference formulation is determined using qPCR. In certain embodiments, the titer of the reference AAV vector particles in the first formulation is determined using ddPCR.

In certain embodiments, the step of measuring the amount of payload molecule produced from the collection of reference AAV vector particles includes: lysing the target cells and collecting the resulting cell lysate sample; adding a molecule of interest to the cell lysate sample, wherein the molecule of interest interacts with the payload molecule to produce a product molecule; and measuring the amount of product molecule produced in the cell lysate, such that the potency of the reference AAV vector particles from reference first formulation is measured. In certain embodiments, the the amount of product molecule produced is measured using Ultra High-Pressure Liquid Chromatography (UHPLC).

In certain embodiments, the titer of the AAV vector particles in the first formulation is determined using qPCR; and the titer of the reference AAV vector particles in the reference formulation is determined using ddPCR. In certain embodiments, the titer of the AAV vector particles in the first formulation is determined using ddPCR; and the titer of the reference AAV vector particles in the reference formulation is determined using qPCR.

In certain embodiments, the target cells are HT1080 cells. In certain embodiments, the HT1080 cells are plated onto a testing plate at a density of 1×10⁴ cells/well.

### DETAILED DESCRIPTION

### I. Adeno-Associated Viruses (AAVs)

### Overview

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety as related to parvoviruses.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

### AAV Viral Genomes

The wild-type AAV viral genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically includes two ITR sequences. These ITRs have a characteristic T-shaped hairpin structure defined by a self-complementary region (145nt in wild-type AAV) at the 5′ and 3′ ends of the ssDNA which form an energetically stable double stranded region. The double stranded hairpin structures include multiple functions including, but not limited to, acting as an origin for DNA replication by functioning as primers for the endogenous DNA polymerase complex of the host viral replication cell.

The wild-type AAV viral genome further includes nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety as related to AAV9/hu.14) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically includes a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence including a payload region with at least one ITR region. Typically, in recombinant AAV viral genomes there are two ITR regions. The rep/cap sequences can be provided in trans during production to generate AAV particles.

In addition to the encoded heterologous payload, AAV vectors may include the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety as related to AAV variants and equivalents.

In certain embodiments, AAV particles, viral genomes and/or payloads of the present disclosure, and the methods of their use, may be as described in WO2017189963, the contents of which are herein incorporated by reference in their entirety as related to AAV particles, viral genomes and/or payloads. AAV particles of the present disclosure may be formulated in any of the gene therapy formulations of the disclosure including any variations of such formulations apparent to those skilled in the art. The reference to “AAV particles”, “AAV particle formulations” and “formulated AAV particles” in the present application refers to the AAV particles which may be formulated and those which are formulated without limiting either.

In certain embodiments, AAV particles of the present disclosure are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e. payload) for delivery to a cell, a tissue, an organ or an organism.

In certain embodiments, the viral genome of the AAV particles of the present disclosure includes at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics include a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV particles of the present disclosure may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the cell.

In certain embodiments, the AAV viral genome of the present disclosure is a scAAV. In certain embodiments, the AAV viral genome of the present disclosure is a ssAAV.

Methods for producing and/or modifying AAV particles are disclosed in the art, such as pseudotyped AAV particles (PCT Patent Publication Nos. WO200028004; WO200123001; WO2004112727; WO 2005005610 and WO 2005072364, the content of each of which is incorporated herein by reference in its entirety as related to producing and/or modifying AAV particles).

AAV particles may be modified to enhance the efficiency of delivery. Such modified AAV particles can be packaged efficiently and be used to successfully infect the target cells at high frequency and with minimal toxicity. In certain embodiments the capsids of the AAV particles are engineered according to the methods described in US Publication Number US 20130195801, the contents of which are incorporated herein by reference in their entirety as related to modifying AAV particles to enhance the efficiency of delivery.

In certain embodiments, the AAV particles including a payload region encoding a polypeptide or protein of the present disclosure, and may be introduced into mammalian cells.

### Inverted Terminal Repeats (ITRs)

The AAV particles of the present disclosure include a viral genome with at least one ITR region and a payload region. In certain embodiments, the viral genome has two ITRs. These two ITRs flank the payload region at the 5′ and 3′ ends. The ITRs function as origins of replication including recognition sites for replication. ITRs include sequence regions which can be complementary and symmetrically arranged. ITRs incorporated into viral genomes of the present disclosure may be included of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome including two ITRs. In certain embodiments, the ITRs are of the same serotype as one another. In another embodiment, the ITRs are of different serotypes. Non-limiting examples include zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length, and those having at least 95% identity thereto.

In certain embodiments, each ITR may be 141 nucleotides in length. In certain embodiments, each ITR may be 130 nucleotides in length. In certain embodiments, each ITR may be 119 nucleotides in length.

In certain embodiments, the AAV particles include two ITRs and one ITR is 141 nucleotides in length and the other ITR is 130 nucleotides in length. In certain embodiments, the AAV particles include two ITRs and both ITR are 141 nucleotides in length.

### Promoters

In certain embodiments, the payload region of the viral genome includes at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety as related to payload/transgene enhancer elements). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of the polypeptides of the present disclosure in a target cell may require a specific promoter, including but not limited to, a promoter that is species specific, inducible, tissue-specific, or cell cycle-specific (see Parr et al., Nat. Med.3:1145-9 (1997); the contents of which are herein incorporated by reference in their entirety as related to polypeptide expression promoters).

In certain embodiments, the promoter is deemed to be efficient when it drives expression of the polypeptide(s) encoded in the payload region of the viral genome of the AAV particle. In certain embodiments, the promoter is a promoter deemed to be efficient when it drives expression in the cell being targeted. In certain embodiments, the promoter is a promoter having a tropism for the cell being targeted. In certain embodiments, the promoter is a promoter having a tropism for a viral production cell.

In certain embodiments, the promoter drives expression of the payload for a period of time in targeted cells or tissues. Expression driven by a promoter may be for a period of 1-31 days (or any value or range therein), 1-23 months (or any value or range therein), 2-10 years (or any value or range therein), or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter can be a weak promoter for sustained expression of a payload in nervous (e.g. CNS) cells or tissues.

In certain embodiments, the promoter drives expression of the polypeptides of the present disclosure for at least 1-11 months (or any individual value therein), 2-65 years (or any individual value therein), or more than 65 years.

Promoters may be naturally occurring or non-naturally occurring. Non-limiting examples of promoters include viral promoters, plant promoters and mammalian promoters. In certain embodiments, the promoters may be human promoters. In certain embodiments, the promoter may be truncated or mutated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons or subtypes of neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US 20110212529, the contents of which are herein incorporated by reference in their entirety as related to muscle-specific promoters)

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In certain embodiments, the promoter may be less than 1 kb. The promoter may have a length of 200-800 nucleotides (or any value or range therein), or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In certain embodiments, the promoter may be a combination of two or more components of the same or different starting or parental promoters such as, but not limited to, CMV and CBA. Each component may have a length of 200-800 nucleotides (or any value or range therein), or more than 800 nucleotides. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In certain embodiments, the promoter is a combination of a 382 nucleotide CMV-enhancer sequence and a 260 nucleotide CBA-promoter sequence.

In certain embodiments, the viral genome includes a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, EFIα, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EFIa promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HβH construct with a hGUSB promoter, a HSV-1LAT promoter and an NSE promoter and found that the HβH construct showed weaker expression than NSE in mouse brain. Passini and Wolfe (J. Virol. 2001, 12382-12392, the contents of which are herein incorporated by reference in its entirety) evaluated the long term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. SCN8A is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A, Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are herein incorporated by reference in their entireties).

Any of the promoters taught by the aforementioned Yu, Soderblom, Gill, Husain, Passini, Xu, Drews or Raymond may be used in the present disclosures.

In certain embodiments, the promoter is not cell specific.

In certain embodiments, the promoter is a ubiquitin c (UBC) promoter. The UBC promoter may have a size of 300-350 nucleotides. As a non-limiting example, the UBC promoter is 332 nucleotides. In certain embodiments, the promoter is a β-glucuronidase (GUSB) promoter. The GUSB promoter may have a size of 350-400 nucleotides. As a non-limiting example, the GUSB promoter is 378 nucleotides. In certain embodiments, the promoter is a neurofilament light (NFL) promoter. The NFL promoter may have a size of 600-700 nucleotides. As a non-limiting example, the NFL promoter is 650 nucleotides. In certain embodiments, the promoter is a neurofilament heavy (NFH) promoter. The NFH promoter may have a size of 900-950 nucleotides. As a non-limiting example, the NFH promoter is 920 nucleotides. In certain embodiments, the promoter is a SCN8A promoter. The SCN8A promoter may have a size of 450-500 nucleotides. As a non-limiting example, the SCN8A promoter is 470 nucleotides.

In certain embodiments, the promoter is a frataxin (FXN) promoter. In certain embodiments, the promoter is a phosphoglycerate kinase 1 (PGK) promoter. In certain embodiments, the promoter is a chicken β-actin (CBA) promoter, or variant thereof. In certain embodiments, the promoter is a CB6 promoter. In certain embodiments, the promoter is a minimal CB promoter. In certain embodiments, the promoter is a cytomegalovirus (CMV) promoter. In certain embodiments, the promoter is a H1 promoter. In certain embodiments, the promoter is a CAG promoter. In certain embodiments, the promoter is a GFAP promoter. In certain embodiments, the promoter is a synapsin promoter. In certain embodiments, the promoter is an engineered promoter. In certain embodiments, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12. In certain embodiments, the promoter is a RNA pol III promoter. As a non-limiting example, the RNA pol III promoter is U6. As a non-limiting example, the RNA pol III promoter is H1. In certain embodiments, the promoter is a cardiomyocyte-specific promoter. Non-limiting examples of cardiomyocyte-specific promoters include αMHC, cTnT, and CMV-MLC2k. In certain embodiments, the viral genome includes two promoters. As a non-limiting example, the promoters are an EF1α promoter and a CMV promoter.

In certain embodiments, the viral genome includes an enhancer element, a promoter and/or a 5′ UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′ UTR/intron may be, but is not limited to, SV40, and CBA-MVM. As a non-limiting example, the enhancer, promoter and/or intron used in combination may be: (1) CMV enhancer, CMV promoter, SV40 5′ UTR intron; (2) CMV enhancer, CBA promoter, SV 40 5′ UTR intron; (3) CMV enhancer, CBA promoter, CBA-MVM 5′ UTR intron; (4) UBC promoter; (5) GUSB promoter; (6) NSE promoter; (7) Synapsin promoter; (8) MeCP2 promoter and (9) GFAP promoter.

In certain embodiments, the viral genome includes an engineered promoter.

In another embodiment, the viral genome includes a promoter from a naturally expressed protein.

### Payload

AAV particles of the present disclosure can include, or be produced using, at least one payload construct which includes at least one payload region. In certain embodiments, the payload region may be located within a viral genome, such as the viral genome of a payload construct. At the 5′ and/or the 3′ end of the payload region there may be at least one inverted terminal repeat (ITR). Within the payload region, there may be a promoter region, an intron region and a coding region.

In certain embodiments, a payload construct of the present disclosure can be a bacmid, also known as a baculovirus plasmid or recombinant baculovirus genome.

In certain embodiments, the payload region of the AAV particle includes one or more nucleic acid sequences encoding a polypeptide or protein of interest.

In certain embodiments, the AAV particle includes a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the vector genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which include the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the AAV particle includes a viral genome with a payload region comprising a nucleic acid sequence encoding a protein of interest (i.e. a payload protein, therapeutic protein).

In certain embodiments, the payload region comprises a nucleic acid sequence encoding a protein including but not limited to an antibody, Aromatic L-Amino Acid Decarboxylase (AADC), ApoE2, Frataxin, survival motor neuron (SMN) protein, glucocerebrosidase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1) and/or gigaxonin (GAN).

In certain embodiments, the AAV particle includes a viral genome with a payload region comprising a nucleic acid sequence encoding any of the disease-associated proteins (and fragment or variants thereof) described in any one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety insofar as they do no conflict with the present disclosure.

Amino acid sequences encoded by payload regions of the viral genomes of the disclosure may be translated as a whole polypeptide, a plurality of polypeptides or fragments of polypeptides, which independently may be encoded by one or more nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

In certain embodiments a “polypeptide variant” is provided. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and in certain embodiments, they will be at least about 80%, or at least about 90% identical (homologous) to a native or reference sequence.

The present disclosure comprises the use of formulated AAV particles whose vector genomes encode modulatory polynucleotides, e.g., RNA or DNA molecules as therapeutic agents. Accordingly, the present disclosure provides vector genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle includes a viral genome with a payload region comprising a nucleic acid sequence encoding or including one or more modulatory polynucleotides. In certain embodiments, the AAV particle includes a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, the payload region comprises a nucleic acid sequence encoding a modulatory polynucleotide which interferes with a target gene expression and/or a target protein production. In certain embodiments, the gene expression or protein production to be inhibited/modified may include but are not limited to superoxide dismutase 1 (SOD1), chromosome 9 open reading frame 72 (C9ORF72), TAR DNA binding protein (TARDBP), ataxin-3 (ATXN3), huntingtin (HTT), amyloid precursor protein (APP), apolipoprotein E (ApoE), microtubule-associated protein tau (MAPT), alpha-synuclein (SNCA), voltage-gated sodium channel alpha subunit 9 (SCN9A), and/or voltage-gated sodium channel alpha subunit 10 (SCN10A).

In certain embodiments, the AAV particle includes a viral genome with a payload region comprising a nucleic acid sequence encoding any of the modulatory polynucleotides, RNAi molecules, siRNA molecules, dsRNA molecules, and/or RNA duplexes described in any one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety insofar as they do no conflict with the present disclosure.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, for 2 nucleotide overhangs at the 3′end of each strand.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases it requires higher concentration of the ss-siRNA to achieve the effective gene silencing potency of the corresponding duplex.

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) includes molecular scaffold which comprises a leading 5′ flanking sequence which may be of any length and may be derived in whole or in part from wild type microRNA sequence or be completely artificial. A 3′ flanking sequence may mirror the 5′ flanking sequence in size and origin. In certain embodiments, one or both of the 5′ and 3′ flanking sequences are absent.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

### Genome Size

In certain embodiments, the AAV particle which includes a payload described herein may be single stranded or double stranded vector genome. The size of the vector genome may be small, medium, large or the maximum size. Additionally, the vector genome may include a promoter and a polyA tail.

In certain embodiments, the vector genome which includes a payload described herein may be a small single stranded vector genome. A small single stranded vector genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded vector genome may be 3.2 kb in size. As another non-limiting example, the small single stranded vector genome may be 2.2 kb in size. Additionally, the vector genome may include a promoter and a polyA tail.

In certain embodiments, the vector genome which includes a payload described herein may be a small double stranded vector genome. A small double stranded vector genome may be 1.3 to 1.7 kb in size such as about 1.3, 1.4, 1.5, 1.6, and 1.7 kb in size. As a non-limiting example, the small double stranded vector genome may be 1.6 kb in size. Additionally, the vector genome may include a promoter and a polyA tail.

In certain embodiments, the vector genome which includes a payload described herein e.g., polynucleotide, siRNA or dsRNA, may be a medium single stranded vector genome. A medium single stranded vector genome may be 3.6 to 4.3 kb in size such as about 3.6, 3.7, 3.8, 3.9, 4.0, 4.1, 4.2 and 4.3 kb in size. As a non-limiting example, the medium single stranded vector genome may be 4.0 kb in size. Additionally, the vector genome may include a promoter and a polyA tail.

In certain embodiments, the vector genome which includes a payload described herein may be a medium double stranded vector genome. A medium double stranded vector genome may be 1.8 to 2.1 kb in size such as about 1.8, 1.9, 2.0, and 2.1 kb in size. As a non-limiting example, the medium double stranded vector genome may be 2.0 kb in size. Additionally, the vector genome may include a promoter and a polyA tail.

In certain embodiments, the vector genome which includes a payload described herein may be a large single stranded vector genome. A large single stranded vector genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded vector genome may be 4.7 kb in size. As another non-limiting example, the large single stranded vector genome may be 4.8 kb in size. As yet another non-limiting example, the large single stranded vector genome may be 6.0 kb in size. Additionally, the vector genome may include a promoter and a polyA tail.

In certain embodiments, the vector genome which includes a payload described herein may be a large double stranded vector genome. A large double stranded vector genome may be 2.2 to 3.0 kb in size such as about 2.2, 2.3, 2.4, 2.5, 2.6, 2.7, 2.8, 2.9 and 3.0 kb in size. As a non-limiting example, the large double stranded vector genome may be 2.4 kb in size. Additionally, the vector genome may include a promoter and a polyA tail.

### AAV Serotypes

AAV particles of the present disclosure may include or be derived from any natural or recombinant AAV serotype. According to the present disclosure, the AAV particles may utilize or be based on a serotype or include a peptide selected from any of the following: VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAVS, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAVS-3/rh.57, AAVS-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVCS, AAV-DJ, AAV-DJ8, AAVF3, AAVFS, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T , AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101 , AAV-8h, AAV-8b, AAV-h, AAV-b, AAV SM 10-2 , AAV Shuffle 100-1 , AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8 , AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clvl-10, AAV CLv1-2, AAV CLv-12, AAV CLv1-3, AAV CLv-13, AAV CLv1-4, AAV Clvl-7, AAV Clvl-8, AAV Clvl-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAVrh20, AAVrh32/33, AAVrh39, AAVrh46, AAVrh73, AAVrh74, AAVhu.26, or variants or derivatives thereof.

The AAV-DJ sequence may include two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may include three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In certain embodiments, the AAV may be a serotype generated by the AAV9 capsid library with mutations in amino acids 390-627 (VP1 numbering) The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A,G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; 0 (Pyl) for Pyrrolysine; U (Sec) for Selenocysteine; X (Xaa) for any amino acid; and Z (Glx) for Glutamine or Glutamic acid.

In certain embodiments, the AAV serotype may be, or may include a sequence, insert, modification or mutation as described in Patent Publications WO2015038958, WO2017100671, WO2016134375, WO2017083722, WO2017015102, WO2017058892, WO2017066764, U.S. Pat. No. 9,624,274, U.S. Pat. No. 9,475,845, US20160369298, US20170145405, the contents of which are herein incorporated by reference in their entirety as related to AAV serotypes and modifications.

In certain embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In certain embodiments, the AAV serotype may be as described in Jackson et al (Frontiers in Molecular Neuroscience 9:154 (2016)), the contents of which are herein incorporated by reference in their entirety AAV serotypes and modifications.

In certain embodiments, the AAV serotype is selected for use due to its tropism for cells of the central nervous system. In certain embodiments, the cells of the central nervous system are neurons. In another embodiment, the cells of the central nervous system are astrocytes.

In certain embodiments, the AAV serotype is selected for use due to its tropism for cells of the muscle(s).

In certain embodiments, the initiation codon for translation of the AAV VP1 capsid protein may be CTG, TTG, or GTG as described in U.S. Pat. No. 8,163,543, the contents of which are herein incorporated by reference in its entirety AAV serotypes and modifications.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins including the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety as related to capsid protein modification and clipping.

According to the present disclosure, references to capsid proteins is not limited to either clipped (Met−/AA−) or unclipped (Met+/AA+) and may, in context, refer to independent capsid proteins, viral capsids included of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also include VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which includes or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Introduction Into Cells

The encoded siRNA molecules (e.g., siRNA duplexes) of the present disclosure may be introduced into cells by being encoded by the vector genome of an AAV particle. These AAV particles are engineered and optimized to facilitate the entry into cells that are not readily amendable to transfection/transduction. Also, some synthetic viral vectors possess an ability to integrate the shRNA into the cell genome, thereby leading to stable siRNA expression and long-term knockdown of a target gene. In this manner, viral vectors are engineered as vehicles for specific delivery while lacking the deleterious replication and/or integration features found in wild-type virus.

In certain embodiments, the encoded siRNA molecule is introduced into a cell by transfecting, infecting or transducing the cell with an AAV particle comprising nucleic acid sequences capable of producing the siRNA molecule when transcribed in the cell. In certain embodiments, the siRNA molecule is introduced into a cell by injecting into the cell or tissue an AAV particle comprising a nucleic acid sequence capable of producing the siRNA molecule when transcribed in the cell.

In certain embodiments, prior to transfection/transduction, an AAV particle comprising a nucleic acid sequence encoding the siRNA molecules of the present disclosure may be transfected into cells.

Other methods for introducing AAV particles comprising the nucleic acid sequence for the siRNA molecules described herein may include photochemical internalization as described in U. S. Patent publication No. 20120264807; the content of which is herein incorporated by reference in its entirety as related to photochemical internalizations.

In certain embodiments, the formulations described herein may contain at least one AAV particle comprising the nucleic acid sequence encoding the siRNA molecules described herein. In certain embodiments, the siRNA molecules may target the gene of interest at one target site. In another embodiment, the formulation comprises a plurality of AAV particles, each AAV particle comprising a nucleic acid sequence encoding a siRNA molecule targeting the gene of interest at a different target site. The gene of interest may be targeted at 2, 3, 4, 5 or more than 5 sites.

In certain embodiments, the AAV particles from any relevant species, such as, but not limited to, human, pig, dog, mouse, rat or monkey may be introduced into cells.

In certain embodiments, the formulated AAV particles may be introduced into cells or tissues which are relevant to the disease to be treated.

In certain embodiments, the formulated AAV particles may be introduced into cells which have a high level of endogenous expression of the target sequence.

In another embodiment, the formulated AAV particles may be introduced into cells which have a low level of endogenous expression of the target sequence.

In certain embodiments, the cells may be those which have a high efficiency of AAV transduction.

In certain embodiments, formulated AAV particles comprising a nucleic acid sequence encoding the siRNA molecules of the present disclosure may be used to deliver siRNA molecules to the central nervous system (e.g., U.S. Pat. No. 6,180,613; the contents of which is herein incorporated by reference in its entirety as related to the deliver and therapeutic use of siRNA molecules and AAV particles).

In certain embodiments, the formulated AAV particles comprising a nucleic acid sequence encoding the siRNA molecules of the present disclosure may further comprise a modified capsid including peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In certain embodiments, the formulated AAV particle comprising a nucleic acid sequence encoding the siRNA molecules of the present disclosure may encode siRNA molecules which are polycistronic molecules. The siRNA molecules may additionally comprise one or more linkers between regions of the siRNA molecules.

In certain embodiments, a formulated AAV particle may comprise at least one of the modulatory polynucleotides encoding at least one of the siRNA sequences or duplexes described herein.

In certain embodiments, an expression vector may comprise, from ITR to ITR recited 5′ to 3′, an ITR, a promoter, an intron, a modulatory polynucleotide, a polyA sequence and an ITR.

In certain embodiments, the encoded siRNA molecule may be located downstream of a promoter in an expression vector such as, but not limited to, CMV, U6, H1, CBA or a CBA promoter with a SV40 intron. Further, the encoded siRNA molecule may also be located upstream of the polyadenylation sequence in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In certain embodiments, the encoded siRNA molecule may be located upstream of the polyadenylation sequence in an expression vector. Further, the encoded siRNA molecule may be located downstream of a promoter such as, but not limited to, CMV, U6, CBA or a CBA promoter with a SV40 intron in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In certain embodiments, the encoded siRNA molecule may be located in a scAAV.

In certain embodiments, the encoded siRNA molecule may be located in an ssAAV.

In certain embodiments, the encoded siRNA molecule may be located near the 5′ end of the flip ITR in an expression vector. In another embodiment, the encoded siRNA molecule may be located near the 3′ end of the flip ITR in an expression vector. In yet another embodiment, the encoded siRNA molecule may be located near the 5′ end of the flop ITR in an expression vector. In yet another embodiment, the encoded siRNA molecule may be located near the 3′ end of the flop ITR in an expression vector. In certain embodiments, the encoded siRNA molecule may be located between the 5′ end of the flip ITR and the 3′ end of the flop ITR in an expression vector. In certain embodiments, the encoded siRNA molecule may be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the 5′ or 3′ end of an ITR (e.g., Flip or Flop ITR) in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides upstream from the 5′ or 3′ end of an ITR (e.g., Flip or Flop ITR) in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the 5′ or 3′ end of an ITR (e.g., Flip or Flop ITR) in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 upstream from the 5′ or 3′ end of an ITR (e.g., Flip or Flop ITR) in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides upstream from the 5′ or 3′ end of an ITR (e.g., Flip or Flop ITR) in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the 5′ or 3′ end of an ITR (e.g., Flip or Flop ITR) in an expression vector.

In certain embodiments, AAV particle comprising the nucleic acid sequence for the siRNA molecules of the present disclosure may be formulated for CNS delivery. Agents that cross the brain blood barrier may be used. For example, some cell penetrating peptides that can target siRNA molecules to the brain blood barrier endothelium may be used to formulate the siRNA duplexes targeting the gene of interest.

In certain embodiments, the formulated AAV particle comprising a nucleic acid sequence encoding the siRNA molecules of the present disclosure may be administered directly to the CNS. As a non-limiting example, the vector comprises a nucleic acid sequence encoding the siRNA molecules targeting the gene of interest.

In specific embodiments, compositions of formulated AAV particles comprising a nucleic acid sequence encoding the siRNA molecules of the present disclosure may be administered in a way which facilitates the vectors or siRNA molecule to enter the central nervous system and penetrate into motor neurons.

In certain embodiments, the formulated AAV particle may be administered to a subject (e.g., to the CNS of a subject via intrathecal administration) in a therapeutically effective amount for the siRNA duplexes or dsRNA to target the motor neurons and astrocytes in the spinal cord and/or brain stem. As a non-limiting example, the siRNA duplexes or dsRNA may reduce the expression of a protein or mRNA.

### II. AAV Production

### General Viral Production Process

Viral production cells for the production of rAAV particles generally include mammalian cell types. However, mammalian cells present several complications to the large-scale production of rAAV particles, including general low yield of viral-particles-per-replication-cell as well as high risks for undesirable contamination from other mammalian biomaterials in the viral production cell. As a result, insect cells have become an alternative vehicle for large-scale production of rAAV particles.

AAV production systems using insect cells also present a range of complications. For example, high-yield production of rAAV particles often requires a lower expression of Rep78 compared to Rep52. Controlling the relative expression of Rep78 and Rep52 in insect cells thus requires carefully designed control mechanisms within the Rep operon. These control mechanisms can include individually engineered insect cell promoters, such as AIE1 promoters for Rep78 and PolH promoters for Rep52, or the division of the Rep-encoding nucleotide sequences onto independently engineered sequences or constructs. However, implementation of these control mechanisms often leads to reduced rAAV particle yield or to structurally unstable virions.

In another example, production of rAAV particles requires VP1, VP2 and VP3 proteins which assemble to form the AAV capsid. High-yield production of rAAV particles requires adjusted ratios of VP1, VP2 and VP3, which should generally be around 1:1:10, respectively, but can vary from 1-2 for VP1 and/or 1-2 for VP2, relative to 10 VP3 copies. This ratio is important for the quality of the capsid, as too much VP1 destabilizes the capsid and too little VP1 will decrease the infectivity of the virus.

Wild type AAV use a deficient splicing method to control VP1 expression; a weak start codon (ACG) with special surrounding (“Kozak” sequence) to control VP2; and a standard start codon (ATG) for VP3 expression. However, in some baculovirus systems, the mammalian splicing sequences are not always recognized and unable to properly control the production of VP1, VP2 and VP3. Consequently, neighboring nucleotides and the ACG start sequence from VP2 can be used to drive capsid protein production. Unfortunately, for most of the AAV serotypes, this method creates a capsid with a lower ratio of VP1 compared to VP2 (<1 relative to 10 VP3 copies). To more effectively control the production of VP proteins, non-canonical or start codons have been used, like TTG, GTG or CTG. However, these start codons are considered suboptimal by those in the art relative to the wild type ATG or ACG start codons (See, WO2007046703 and WO2007148971, the contents of which are incorporated herein by reference in their entirety as related to production of AAV capsid proteins).

In another example, production of rAAV particles using a baculovirus/Sf9 system generally requires the widely used bacmid-based Baculovirus Expression Vector System (BEVs), which are not optimized for large-scale AAV production. Aberrant proteolytic degradation of viral proteins in the bacmid-based BEVs is an unexpected issue, precluding the reliable large-scale production of AAV capsid proteins using the baculovirus/Sf9 system.

There is continued need for methods and systems which allow for effective and efficient large scale (commercial) production of rAAV particles in mammalian and insect cells.

The details of one or more embodiments of the present disclosure are set forth in the accompanying description below. Other features, objects, and advantages of the present disclosure will be apparent from the description, drawings, and the claims. In the description, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict with disclosures incorporated by reference, the present express description will control.

In certain embodiments, the constructs, polynucleotides, polypeptides, vectors, serotypes, capsids formulations, or particles of the present disclosure may be, may include, may be modified by, may be used by, may be used for, may be used with, or may be produced with any sequence, element, construct, system, target or process described in one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety insofar as they do no conflict with the present disclosure.

AAV production of the present disclosure includes processes and methods for producing AAV particles and viral vectors which can contact a target cell to deliver a payload, e.g. a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

The present disclosure provides methods of producing AAV particles or viral vectors by contacting a viral production cell with one or more viral production constructs. Viral production constructs can include viral expression constructs and payload constructs.

The present disclosure provides methods of producing AAV particles or viral vectors by (a) contacting a viral production cell with one or more viral expression constructs encoding at least one chimeric capsid protein, and one or more payload construct vectors, wherein said payload construct vector includes a payload construct encoding a payload molecule selected from the group consisting of a transgene, a polynucleotide encoding protein, and a modulatory nucleic acid; (b) culturing said viral production cell under conditions such that at least one AAV particle or viral vector is produced, and (c) isolating said at least one AAV particle or viral vector.

In these methods a viral expression construct may encode at least one structural protein and/or at least one non-structural protein. The structural protein may include any of the native or wild type capsid proteins VP1, VP2 and/or VP3 or a chimeric protein. The non-structural protein may include any of the native or wild type Rep78, Rep68, Rep52 and/or Rep40 proteins or a chimeric protein.

In certain embodiments, contacting occurs via transient transfection, viral transduction and/or electroporation.

In certain embodiments, the viral production cell is selected from the group consisting of a mammalian cell and an insect cell. In certain embodiments, the insect cell includes a Spodoptera frugiperda insect cell. In certain embodiments, the insect cell includes a Sf9 insect cell. In certain embodiments, the insect cell includes a Sf21 insect cell.

The payload construct vector of the present disclosure may include at least one inverted terminal repeat (ITR) and may include mammalian DNA.

Also provided are AAV particles and viral vectors produced according to the methods described herein.

The AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell or a mammalian cell) with at least one viral expression construct encoding at least one capsid protein and at least one payload construct vector. The viral production cell may be contacted by transient transfection, viral transduction and/or electroporation. The payload construct vector may include a payload construct encoding a payload molecule such as, but not limited to, a transgene, a polynucleotide encoding protein, and a modulatory nucleic acid. The viral production cell can be cultured under conditions such that at least one AAV particle or viral vector is produced, isolated (e.g., using temperature-induced lysis, mechanical lysis and/or chemical lysis) and/or purified (e.g., using filtration, chromatography and/or immunoaffinity purification). As a non-limiting example, the payload construct vector may include mammalian DNA.

In certain embodiments, the AAV particles are produced in an insect cell (e.g., Spodoptera frugiperda (Sf9) cell) using the method described herein. As a non-limiting example, the insect cell is contacted using viral transduction which may include baculoviral transduction.

In another embodiment, the AAV particles are produced in a mammalian cell using the method described herein. As a non-limiting example, the mammalian cell is contacted using transient transfection.

In certain embodiments, the viral expression construct may encode at least one structural protein and at least one non-structural protein. As a non-limiting example, the structural protein includes VP1, VP2 and/or VP3. As another non-limiting example, the non-structural protein includes Rep78, Rep68, Rep52 and/or Rep40.

In certain embodiments, the AAV particle production method described herein produces greater than 10¹, greater than 10², greater than 10³, greater than 10⁴ or greater than 10⁵ AAV particles in a viral production cell.

In certain embodiments, a process of the present disclosure includes production of viral particles in a viral production cell using a viral production system which includes at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, including Rep proteins which replicate the payload construct and Cap proteins which assemble to form a capsid that encloses the replicated payload constructs. The resulting AAV particle is extracted from the viral production cells and processed into a pharmaceutical preparation for administration.

Once administered, the AAV particles contacts a target cell and enters the cell in an endosome. The AAV particle releases from the endosome and subsequently contacts the nucleus of the target cell to deliver the payload construct. The payload construct, e.g. recombinant viral construct, is delivered to the nucleus of the target cell wherein the payload molecule encoded by the payload construct may be expressed.

In certain embodiments, the process for production of viral particles utilizes seed cultures of viral production cells that include one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naive population of production cells.

Large scale production of AAV particles may utilize a bioreactor. The use of a bioreactor allows for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In another embodiment, the bioreactor is used for continuous production in which a portion of the culture is harvested at an experimentally determined time point for purification of AAV particles, and the remaining culture in the bioreactor is refreshed with additional growth media components.

AAV viral particles can be extracted from viral production cells in a process which includes cell lysis, clarification, sterilization and purification. Cell lysis includes any process that disrupts the structure of the viral production cell, thereby releasing AAV particles. In certain embodiments cell lysis may include thermal shock, chemical, or mechanical lysis methods. Clarification can include the gross purification of the mixture of lysed cells, media components, and AAV particles. In certain embodiments, clarification includes centrifugation and/or filtration, including but not limited to depth end, tangential flow, and/or hollow fiber filtration.

The end result of viral production is a purified collection of AAV particles which include two components: (1) a payload construct (e.g. a recombinant viral genome construct) and (2) a viral capsid.

In certain embodiments, a viral production system or process of the present disclosure includes steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can include a single CP Purification step or can include multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure includes steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs and Payload BIICs, with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

### Viral Expression Constructs

The viral production system of the present disclosure includes one or more viral expression constructs which can be transfected/transduced into a viral production cell. In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be a parvoviral plasmid. In certain embodiments, the viral expression includes a protein-coding nucleotide sequence and at least one expression control sequence for expression in a viral production cell. In certain embodiments, the viral expression includes a protein-coding nucleotide sequence operably linked to least one expression control sequence for expression in a viral production cell. In certain embodiments, the viral expression construct contains parvoviral genes under control of one or more promoters. Parvoviral genes can include nucleotide sequences encoding non-structural AAV replication proteins, such as Rep genes which encode Rep52, Rep40, Rep68 or Rep78 proteins. Parvoviral genes can include nucleotide sequences encoding structural AAV proteins, such as Cap genes which encode VP1, VP2 and VP3 proteins.

In certain embodiments, a viral expression construct can include a Rep52-coding region; a Rep52-coding region is a nucleotide sequence which includes a Rep52 nucleotide sequence encoding a Rep52 protein. In certain embodiments, a viral expression construct can include a Rep78-coding region; a Rep78-coding region is a nucleotide sequence which includes a Rep78 nucleotide sequence encoding a Rep78 protein. In certain embodiments, a viral expression construct can include a Rep40-coding region; a Rep40-coding region is a nucleotide sequence which includes a Rep40 nucleotide sequence encoding a Rep40 protein. In certain embodiments, a viral expression construct can include a Rep68-coding region; a Rep68-coding region is a nucleotide sequence which includes a Rep68 nucleotide sequence encoding a Rep68 protein.

In certain embodiments, a viral expression construct can include a VP-coding region; a VP-coding region is a nucleotide sequence which includes a VP nucleotide sequence encoding VP1, VP2, VP3, or a combination thereof. In certain embodiments, a viral expression construct can include a VP1-coding region; a VP1-coding region is a nucleotide sequence which includes a VP1 nucleotide sequence encoding a VP1 protein. In certain embodiments, a viral expression construct can include a VP2-coding region; a VP2-coding region is a nucleotide sequence which includes a VP2 nucleotide sequence encoding a VP2 protein. In certain embodiments, a viral expression construct can include a VP3-coding region; a VP3-coding region is a nucleotide sequence which includes a VP3 nucleotide sequence encoding a VP3 protein.

Structural VP proteins, VP1, VP2, and VP3, and non-structural proteins, Rep52 and Rep78, of the viral expression construct can be encoded in a single open reading frame regulated by utilization of both alternative splice acceptor and non-canonical translational initiation codons. Both Rep78 and Rep52 can be translated from a single transcript: Rep78 translation initiates at a first start codon (AUG or non-AUG) and Rep52 translation initiates from a Rep52 start codon (e.g. AUG) within the Rep78 sequence. Rep78 and Rep52 can also be translated from separate transcripts with independent start codons. The Rep52 initiation codons within the Rep78 sequence can be mutated, modified or removed, such that processing of the modified Rep78 sequence will not produce Rep52 proteins.

VP1, VP2 and VP3 can be transcribed and translated from a single transcript in which both in-frame and/or out-of-frame start codons are engineered to control the VP1:VP2:VP3 ratio produced by the nucleotide transcript. In certain embodiments, VP1 can be produced from a sequence which encodes for VP1 only. As use herein, the terms “only for VP1” or “VP1 only” refers to a nucleotide sequence or transcript which encodes for a VP1 capsid protein and: (i) lacks the necessary start codons within the VP1 sequence (i.e. deleted or mutated) for full transcription or translation of VP2 and VP3 from the same sequence; (ii) includes additional codons within the VP1 sequence which prevent transcription or translation of VP2 and VP3 from the same sequence; or (iii) includes a start codon for VP1 (e.g. ATG), such that VP1 is the primary VP protein produced by the nucleotide transcript.

In certain embodiments, VP2 can be produced from a sequence which encodes for VP2 only. As use herein, the terms “only for VP2” or “VP2 only” refers to a nucleotide sequence or transcript which encodes for a VP2 capsid protein and: (i) the nucleotide transcript is a truncated variant of a full VP capsid sequence which encodes only VP2 and VP3 capsid proteins; and (ii) which include a start codon for VP2 (e.g. ATG), such that VP2 is the primary VP protein produced by the nucleotide transcript.

In certain embodiments, VP1 and VP2 can be produced from a sequence which encodes for VP1 and VP2 only. As use herein, the terms “only for VP1 and VP2” or “VP1 and VP2 only” refer to a nucleotide sequence or transcript which encodes for VP1 and VP2 capsid proteins and: (i) lacks the necessary start codons within the VP sequence (i.e. deleted or mutated) for full transcription or translation of VP3 from the same sequence; (ii) includes additional codons within the VP sequence which prevent transcription or translation of VP3 from the same sequence; (iii) includes a start codon for VP1 (e.g. ATG) and VP2 (e.g. ATG), such that VP1 and VP2 are the primary VP protein produced by the nucleotide transcript; or (iv) includes VP1-only nucleotide transcript and a VP2-only nucleotide transcript connected by a linker, such as an IRES region.

The viral production system of the present disclosure is not limited by the viral expression vector used to introduce the parvoviral functions into the virus replication cell. The presence of the viral expression construct in the virus replication cell need not be permanent. The viral expression constructs can be introduced by any means known, for example by chemical treatment of the cells, electroporation, or infection.

Viral expression constructs of the present disclosure may include any compound or formulation, biological or chemical, which facilitates transformation, transfection, or transduction of a cell with a nucleic acid. Exemplary biological viral expression constructs include plasmids, linear nucleic acid molecules, and recombinant viruses including baculovirus. Exemplary chemical vectors include lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposoes as tools in Basic Research and Industry. CRC Press, Ann Arbor, Mich. (1995), the contents of each of which are herein incorporated by reference in its entirety as related to viral expression constructs and uses thereof.

In certain embodiments, the viral expression construct is an AAV expression construct which includes one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In one non-limiting example, five expression constructs may individually encode AAV VP1, AAV VP2, AAV VP3, Rep52, Rep78, and with an accompanying payload construct comprising a payload polynucleotide and at least one AAV ITR. In another embodiment, expression constructs may be employed to express, for example, Rep52 and Rep40, or Rep78 and Rep 68. Expression constructs may include any combination of VP1, VP2, VP3, Rep52/Rep40, and Rep78/Rep68 coding sequences.

In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct can include one or more expression control sequence between protein-coding nucleotide sequences. In certain embodiments, an expression control region can include an IRES sequence region which includes an IRES nucleotide sequence encoding an internal ribosome entry sight (IRES). The internal ribosome entry sight (IRES) can be selected from the group consisting or: FMDV-IRES from Foot-and-Mouth-Disease virus, EMCV-IRES from Encephalomyocarditis virus, and combinations thereof.

In certain embodiments, an expression control region can include a 2A sequence region which comprises a 2A nucleotide sequence encoding a viral 2A peptide. A viral 2A sequence is a relatively short (approximately 20 amino acids) sequence which contains a consensus sequence of: Asp-Val/Ile-Glu-X-Asn-Pro-Gly-Pro. The sequence allows for co-translation of multiple polypeptides within a single open reading frame (ORF). As the ORF is translated, glycine and proline residues with the 2A sequence prevent the formation of a normal peptide bond, which results in ribosomal “skipping” and “self-cleavage” within the polypeptide chain. The viral 2A peptide can be selected from the group consisting of: F2A from Foot-and-Mouth-Disease virus, T2A from Thosea asigna virus, E2A from Equine rhinitis A virus, P2A from Porcine teschovirus-1, BmCPV2A from cytoplasmic polyhedrosis virus, BmIFV 2A from B. mori flacherie virus, and combinations thereof

In certain embodiments, the viral expression construct may contain a nucleotide sequence which includes start codon region, such as a sequence encoding AAV capsid proteins which include one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG).

In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety as related to AAV capsid proteins and the production thereof.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG and GTG, that effects partial exon skipping upon expression in insect cells, as described in U.S. Pat. No. 8,512,981, the contents of which are herein incorporated by reference in their entirety, for example to promote less abundant expression of Rep78 as compared to Rep52, which may in that it promotes high vector yields.

In certain embodiments, the viral expression construct may be a plasmid vector or a baculoviral construct for the expression in insect cells that contains repeating codons with differential codon biases, for example to achieve improved ratios of Rep proteins, e.g. Rep78 and Rep52 thereby improving large scale (commercial) production of viral expression construct and/or payload construct vectors in insect cells, as taught in U.S. Pat. No. 8,697,417, the contents of which are herein incorporated by reference in their entirety as related to AAV replication proteins and the production thereof.

In another embodiment, improved ratios of rep proteins may be achieved using the method and constructs described in U.S. Pat. No 8,642,314, the contents of which are herein incorporated by reference in their entirety as related to AAV replications proteins and the production thereof.

In certain embodiments, the viral expression construct may encode mutant parvoviral Rep polypeptides which have one or more improved properties as compared with their corresponding wild type Rep polypeptide, such as the preparation of higher virus titers for large scale production. Alternatively, they may be able to allow the production of better-quality viral particles or sustain more stable production of virus. In a non-limiting example, the viral expression construct may encode mutant Rep polypeptides with a mutated nuclear localization sequence or zinc finger domain, as described in Patent Application US 20130023034, the contents of which are herein incorporated by reference in their entirety as related to AAV replications proteins and the production thereof

In certain embodiments, the viral expression construct may encode the components of a Parvoviral capsid with incorporated Gly-Ala repeat region, which may function as an immune invasion sequence, as described in US Patent Application 20110171262, the contents of which are herein incorporated by reference in its entirety as related to Parvoviral capsid proteins.

In certain embodiments of the present disclosure, a viral expression construct may be used for the production of AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g. bacmid) can include a polynucleotide incorporated by homologous recombination (transposon donor/acceptor system) into the bacmid by standard molecular biology techniques known and performed by a person skilled in the art.

In certain embodiments, the polynucleotide incorporated into the bacmid (i.e. polynucleotide insert) can include an expression control sequence operably linked to a protein-coding nucleotide sequence. In certain embodiments, the polynucleotide incorporated into the bacmid can include an expression control sequence which includes a promoter, such as p10 or polH, and which is operably linked to a nucleotide sequence which encodes a structural AAV capsid protein (e.g. VP1, VP2, VP3 or a combination thereof). In certain embodiments, the polynucleotide incorporated into the bacmid can include an expression control sequence which includes a promoter, such as p10 or polH, and which is operably linked to a nucleotide sequence which encodes a non-structural AAV capsid protein (e.g. Rep78, Rep52, or a combination thereof).

In certain embodiments, the polynucleotide insert can be incorporated into the bacmid at the location of a baculoviral gene. In certain embodiments, the polynucleotide insert can be incorporated into the bacmid at the location of a non-essential baculoviral gene. In certain embodiments, the polynucleotide insert can be incorporated into the bacmid by replacing a baculoviral gene or a portion of the baculoviral gene with the polynucleotide insert. In certain embodiments, the polynucleotide insert can be incorporated into the bacmid by replacing a baculoviral gene or a portion of the baculoviral gene with a fusion-polynucleotide which includes the polynucleotide insert and the baculoviral gene (or portion thereof) being replaced.

In certain embodiments, the polynucleotide insert can be incorporated into the bacmid by splitting a baculoviral gene with the polynucleotide insert (i.e. the polynucleotide insert is incorporated into the middle of the gene, separating a 5′-portion of the gene from a 3′-portion of the bacmid gene). In certain embodiments, the polynucleotide insert can be incorporated into the bacmid by splitting a baculoviral gene with the fusion-polynucleotide which includes the polynucleotide insert and a portion of the baculoviral gene which was split. In certain embodiments, the 3′ end of the fusion-polynucleotide includes the 5′-portion of the gene that was split, such that the 5′-portion of the gene in the fusion-polynucleotide and the 3′-portion of the gene remaining in the bacmid form a full or functional portion of the baculoviral gene. In certain embodiments, the 5′ end of the fusion-polynucleotide includes the 3′-portion of the gene that was split, such that the 3′-portion of the gene in the fusion-polynucleotide and the 5′-portion of the gene remaining in the bacmid form a full or functional portion of the baculoviral gene. A non-limiting example is presented in Examples 13 and 14, in which fusion-polynucleotides are engineered and produced to include components from the gta gene ORF (full/partial Ac-lef12 promoter, full/partial Ac-gta gene).

In certain embodiments, the polynucleotide can be incorporated into the bacmid at the location of a restriction endonuclease (REN) cleavage site (i.e. REN access point) associated with a baculoviral gene. In certain embodiments, the REN access point in the bacmid is FseI (corresponding with the gta baculovirus gene) (ggccggcc). In certain embodiments, the REN access point in the bacmid is SdaI (corresponding with the DNA polymerase baculovirus gene) (cctgcagg). In certain embodiments, the REN access point in the bacmid is MauBI (corresponding with the lef-4 baculovirus gene) (cgcgcgcg). In certain embodiments, the REN access point in the bacmid is SbfI (corresponding with the gp64/gp67 baculovirus gene) (cctgcagg). In certain embodiments, the REN access point in the bacmid is I-CeuI (corresponding with the v-cath baculovirus gene) (SEQ ID NO: 1). In certain embodiments, the REN access point in the bacmid is AvrII (corresponding with the egt baculovirus gene) (cctagg). In certain embodiments, the REN access point in the bacmid is NheI (gctagc). In certain embodiments, the REN access point in the bacmid is SpeI (actagt). In certain embodiments, the REN access point in the bacmid is BstZ17I (gtatac). In certain embodiments, the REN access point in the bacmid is NcoI (ccatgg). In certain embodiments, the REN access point in the bacmid is MluI (acgcgt).

In certain embodiments where the bacmid is a double-stranded construct, the REN cleavage site can include a cleavage sequence in one strand and the reverse complement of the cleavage sequence (which also functions as a cleavage sequence) in the other strand. A polynucleotide insert (or strand thereof) can thus include a REN cleavage sequence or the reverse complement REN cleavage sequence (which are generally functionally interchangeable). As a non-limiting example, a strand of a polynucleotide insert can include an FseI cleave sequence (ggccggcc) or its reverse complement REN cleavage sequence (ccggccgg).

Polynucleotides can be incorporated into these REN access points by: (i) providing a polynucleotide insert which has been engineered to include a target REN cleavage sequence (e.g. a polynucleotide insert engineered to include FseI REN sequences at both ends of the polynucleotide); (ii) proving a bacmid which includes the target REN access point for polynucleotide insertion (e.g. a variant of the AcMNPV bacmid bMON14272 which includes an FseI cleavage site (ii) digesting the REN-engineered polynucleotide with the appropriate REN enzyme (e.g. using FseI enzyme to digesting the REN-engineering polynucleotide which includes the FseI regions at both ends, to produce a polynucleotide-FseI insert); (iii) digesting the bacmid with the same REN enzyme to produce a single-cut bacmid at the REN access point (e.g. using FseI enzyme to produce a single-cut bacmid at the FseI location); and (iv) ligating the polynucleotide insert into the single-cut bacmid using an appropriate ligation enzyme, such as T4 ligase enzyme. The result is engineered bacmid DNA which includes the engineered polynucleotide insert at the target REN access point.

The insertion process can be repeated one or more times to incorporate other engineered polynucleotide inserts into the same bacmid at different REN access points (e.g. insertion of a first engineered polynucleotide insert at the AvrII REN access point in the egt, followed by insertion of a second engineered polynucleotide insert at the I-CeuI REN access point in the cath gene, and followed by insertion of a third engineered polynucleotide insert at the FseI REN access point in the gta gene).

In certain embodiments, restriction endonuclease (REN) cleavage can be used to remove one or more wild-type genes from a bacmid. In certain embodiments, restriction endonuclease (REN) cleavage can be used to remove one or more engineered polynucleotide insert which has been previously been inserted into the bacmid. In certain embodiments, restriction endonuclease (REN) cleavage can be used to replace one or more engineered polynucleotide inserts with a different engineered polynucleotide insert which includes the same REN cleavage sequences (e.g. an engineered polynucleotide insert at the FseI REN access point can be replaced with a different engineered polynucleotide insert which includes FseI REN cleavage sequences).

In certain embodiments, viral expression constructs may be used that are taught in U.S. Pat. Nos. 8,512,981, 8,163,543, 8,697,417, 8,642,314, US Patent Publication Nos. US20130296532, US20110119777, US20110136227, US20110171262, US20130023034, International Patent Application Nos. PCT/NL2008/050613, PCT/NL2009/050076, PCT/NL2009/050352, PCT/NL2011/050170, PCT/NL2012/050619 and U.S. patent application Ser. No. 14/149,953, the contents of each of which are herein incorporated by reference in their entirety insofar as they do no conflict with the present disclosure.

In certain embodiments, the viral expression construct of the present disclosure may be derived from viral expression constructs taught in U.S. Pat. Nos. 6,468,524, 6,984,517, 7,479,554, 6,855,314, 7,271,002, 6,723,551, US Patent Publication No. 20140107186, U.S. patent application Ser. No. 09/717,789, U.S. Ser. No. 11/936,394, U.S. Ser. No. 14/004,379, European Patent Application EP1082413, EP2500434, EP 2683829, EP1572893 and International Patent Application PCT/US99/11958, PCT/US01/09123, PCT/EP2012/054303, and PCT/US2002/035829 the contents of each of which are herein incorporated by reference in its entirety insofar as they do no conflict with the present disclosure.

In certain embodiments, the viral expression construct may include sequences from Simian species. In certain embodiments, the viral expression construct may contain sequences, including but not limited to capsid and rep sequences from International Patent Applications PCT/US1997/015694, PCT/US2000/033256, PCT/US2002/019735, PCT/US2002/033645, PCT/US2008/013067, PCT/US2008/013066, PCT/US2008/013065, PCT/US2009/062548, PCT/US2009/001344, PCT/US2010/036332, PCT/US2011/061632, PCT/US2013/041565, U.S. application Ser. No. 13/475,535, U.S. Ser. No. 13/896,722, U.S. Ser. No. 10/739,096, U.S. Ser. No. 14/073,979, US Patent Publication Nos.US20010049144, US20120093853, US20090215871, US20040136963, US20080219954, US20040171807, US20120093778, US20080090281, US20050069866, US20100260799, US20100247490,US20140044680, US20100254947, US20110223135, US20130309205, US20120189582, US20130004461, US20130315871, U.S. Pat. Nos. 6,083,716, 7,838,277, 7,344,872, 8,603,459, 8,105,574, 7,247,472, 8,231,880, 8,524,219, 8,470,310, European Patent Application Nos. EP2301582, EP2286841, EP1944043, EP1453543, EP1409748, EP2463362, EP2220217, EP2220241, EP2220242, EP2350269, EP2250255, EP2435559, EP2643465, EP1409748, EP2325298, EP1240345, the contents of each of which is herein incorporated by reference in its entirety insofar as they do no conflict with the present disclosure.

In certain embodiments, viral expression constructs of the present disclosure may include one or more nucleotide sequence from one or more viral construct described in in International Application No. PCT/US2002/025096, PCT/US2002/033629, PCT/US2003/012405, U.S. application Ser. No. 10/291583, U.S. Ser. No. 10/420284, U.S. Pat. No. 7,319,002, US Patent Publication No. US20040191762, US20130045186, US20110263027, US20110151434, US20030138772, US20030207259, European Application No. EP2338900, EP1456419, EP1310571, EP1359217, EP1427835, EP2338900, EP1456419, EP1310571, EP1359217 and U.S. Pat. Nos. 7,235,393 and 8,524,446 insofar as they do no conflict with the present disclosure.

In certain embodiments, the viral expression constructs of the present disclosure may include sequences or compositions described in International Patent Application No. PCT/US1999/025694, PCT/US1999/010096, PCT/US2001/013000, PCT/US2002/25976, PCT/US2002/033631, PCT/US2002/033630, PCT/US2009/041606, PCT/US2012/025550, U.S. Pat. Nos. 8,637,255, 8,637,255, 7,186,552, 7,105,345, 6,759,237, 7,056,502, 7,198,951, 8,318,480, 7,790,449, 7,282,199, US Patent Publication No. US20130059289, US20040057933, US20040057932, US20100278791, US20080050345, US20080050343, US20080008684, US20060204479, US20040057931, US20040052764, US20030013189, US20090227030, US20080075740, US20080075737, US20030228282, US20130323226, US20050014262, U.S. patent application Ser. No. 14/136,331, U.S. Ser. No. 09/076,369, U.S. Ser. No. 10/738,609, European Application No. EP2573170, EP1127150, EP2341068, EP1845163, EP1127150, EP1078096, EP1285078, EP1463805, EP2010178940, US20140004143, EP2359869, EP1453547, EP2341068, and EP2675902, the contents of each of which are herein incorporated by reference in their entirety insofar as they do no conflict with the present disclosure.

In certain embodiments, viral expression construct of the present disclosure may include one or more nucleotide sequence from one or more of those described in U.S. Pat. Nos. 7,186,552, 7,105,345, 6,759,237, 7,056,502, 7,198,951, 8,318,480, 7,790,449, 7,282,199, US Patent Publication No. US20130059289, US20040057933, US20040057932, US20100278791, US20080050345, US20080050343, US20080008684, US20060204479, US20040057931, US20140004143, US20090227030, US20080075740, US20080075737, US20030228282, US20040052764, US20030013189, US20050014262, US20130323226, U.S. patent application Ser. No. 14/136,331, U.S. Ser. No. 10/738,609, European Patent Application Nos. EP1127150, EP2341068, EP1845163, EP1127150, EP1078096, EP1285078, EP2573170, EP1463805, EP2675902, EP2359869, EP1453547, EP2341068, the contents of each of which are incorporated herein by reference in their entirety insofar as they do no conflict with the present disclosure.

In certain embodiments, the viral expression constructs of the present disclosure may include constructs of modified AAVs, as described in International Patent Application No. PCT/US1995/014018, PCT/US2000/026449, PCT/US2004/028817, PCT/US2006/013375, PCT/US2007/010056, PCT/US2010/032158, PCT/US2010/050135, PCT/US2011/033596, U.S. patent application Ser. No. 12/473,917, U.S. Ser. No. 08/331,384, U.S. Ser. No. 09/670,277, U.S. Pat. Nos. 5,871,982, 5,856,152, 6,251,677, 6,387,368, 6,399,385, 7,906,111, European Patent Application No. EP2000103600, European Patent Publication No. EP797678, EP1046711, EP1668143, EP2359866, EP2359865, EP2357010, EP1046711, EP1218035, EP2345731, EP2298926, EP2292780, EP2292779, EP1668143, US20090197338, EP2383346, EP2359867, EP2359866, EP2359865, EP2357010, EP1866422, US20090317417, EP2016174, US Patent Publication Nos. US20110236353, US20070036760, US20100186103, US20120137379, and US20130281516, the contents of each of which are herein incorporated by reference in their entirety insofar as they do no conflict with the present disclosure.

In certain embodiments, the viral expression constructs of the present disclosure may include one or more constructs described in International Application Nos. PCT/US1999/004367, PCT/US2004/010965, PCT/US2005/014556, PCT/US2006/009699, PCT/US2010/032943, PCT/US2011/033628, PCT/US2011/033616, PCT/US2012/034355, US Patent Nos. US8394386, EP1742668, US Patent Publication Nos. US20080241189, US20120046349, US20130195801, US20140031418, EP2425000, US20130101558, EP1742668, EP2561075, EP2561073, EP2699688, the contents of each of which is herein incorporated by reference in its entirety insofar as they do no conflict with the present disclosure.

### Expression Control

### Expression Control Regions

The viral expression constructs of the present disclosure can include one or more expression control region encoded by expression control sequences. In certain embodiments, the expression control sequences are for expression in a viral production cell, such as an insect cell. In certain embodiments, the expression control sequences are operably linked to a protein-coding nucleotide sequence. In certain embodiments, the expression control sequences are operably linked to a VP coding nucleotide sequence or a Rep coding nucleotide sequence.

Herein, the terms “coding nucleotide sequence”, “ protein-encoding gene” or “protein-coding nucleotide sequence” refer to a nucleotide sequence that encodes or is translated into a protein product, such as VP proteins or Rep proteins. “Operably linked” means that the expression control sequence is positioned relative to the coding sequence such that it can promote the expression of the encoded gene product.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can include promoters, enhancers, untranslated regions (UTRs), internal ribosome entry sites (IRES), transcription terminators, a start codon in front of a protein-encoding gene, splicing signal for introns, and stop codons. The term “expression control sequence” is intended to include, at a minimum, a sequence whose presence are designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of an mRNA, sequences referred to as polyA sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

In certain embodiments, the expression control sequence can include one or more promoters. Promoters can include, but are not limited to, baculovirus major late promoters, insect virus promoters, non-insect virus promoters, vertebrate virus promoters, nuclear gene promoters, chimeric promoters from one or more species including virus and non-virus elements, and/or synthetic promoters. In certain embodiments, a promoter can be Ctx, Op-EI, EI, ΔEI, EI-1, pH, PIO, polH (polyhedron), ΔpolH, Dmhsp70, Hr1, Hsp70, 4xHsp27 EcRE+minimal Hsp70, IE, IE-1, ΔIE-1, ΔIE, p10, Δp10 (modified variations or derivatives of p10), p5, p19, p35, p40, p6.9, and variations or derivatives thereof. In certain embodiments, the promoter is a Ctx promoter. In certain embodiments, the promoter is a p10 promoter. In certain embodiments, the promoter is a polH promoter. In certain embodiments, a promoter can be selected from tissue-specific promoters, cell-type-specific promoters, cell-cycle-specific promoters, and variations or derivatives thereof. In certain embodiments, a promoter can be a CMV promoter, an alpha 1-antitrypsin (α1-AT) promoter, a thyroid hormone-binding globulin promoter, a thyroxine-binding globlin (LPS) promoter, an HCR-ApoCII hybrid promoter, an HCR-hAAT hybrid promoter, an albumin promoter, an apolipoprotein E promoter, an α1-AT+EaIb promoter, a tumor-selective E2F promoter, a mononuclear blood IL-2 promoter, and variations or derivatives thereof. In certain embodiments, the promoter is a low-expression promoter sequence. In certain embodiments, the promoter is an enhanced-expression promoter sequence. In certain embodiments, the promoter can include Rep or Cap promoters as described in US Patent Application 20110136227, the contents of which are herein incorporated by reference in its entirety as related to expression promoters.

In certain embodiments, a viral expression construct can include the same promoter in all nucleotide sequences. In certain embodiments, a viral expression construct can include the same promoter in two or more nucleotide sequences. In certain embodiments, a viral expression construct can include a different promoter in two or more nucleotide sequences. In certain embodiments, a viral expression construct can include a different promoter in all nucleotide sequences.

In certain embodiments the viral expression construct encodes elements to improve expression in certain cell types. In a further embodiment, the expression construct may include polh and/or ΔIE-1 insect transcriptional promoters, CMV mammalian transcriptional promoter, and/or p10 insect specific promoters for expression of a desired gene in a mammalian or insect cell.

More than one expression control sequence can be operably linked to a given nucleotide sequence. For example, a promoter sequence, a translation initiation sequence, and a stop codon can be operably linked to a nucleotide sequence.

In certain embodiments, the viral expression construct may contain a nucleotide sequence which includes start codon region, such as a sequence encoding AAV capsid proteins which include one or more start codon regions. In certain embodiments, the start codon region can be within an expression control sequence.

The translational start site of eukaryotic mRNA is controlled in part by a nucleotide sequence referred to as a Kozak sequence as described in Kozak, M Cell. 1986 Jan. 31; 44(2):283-92 and Kozak, M. J Cell Biol. 1989 Feb;108(2):229-41 the contents of each of which are herein incorporated by reference in their entirety as related to Kozak sequences and uses thereof. Both naturally occurring and synthetic translational start sites of the Kozak form can be used in the production of polypeptides by molecular genetic techniques, Kozak, M. Mamm Genome. 1996 August; 7(8):563-74 the contents of which are herein incorporated by reference in their entirety as related to Kozak sequences and uses thereof. Splice sites are sequences on an mRNA which facilitate the removal of parts of the mRNA sequences after the transcription (formation) of the mRNA. Typically, the splicing occurs in the nucleus, prior to mRNA transport into a cell's cytoplasm.

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is 1:1:10 (VP1:VP2:VP3).

In certain embodiments, AAV particles are produced in viral production cells (such as mammalian or insect cells) wherein all three VP proteins are expressed at a stoichiometry approaching, about or which is 2:2:10 (VP1:VP2:VP3).

In certain embodiments, AAV particles are produced in viral production cells (such as mammalian or insect cells) wherein all three VP proteins are expressed at a stoichiometry approaching, about or which is 2:0:10 (VP1:VP2:VP3).

In certain embodiments, AAV particles are produced in viral production cells (such as mammalian or insect cells) wherein all three VP proteins are expressed at a stoichiometry approaching, about or which is 1-2:0-2:10 (VP1:VP2:VP3).

In certain embodiments, AAV particles are produced in viral production cells (such as mammalian or insect cells) wherein all three VP proteins are expressed at a stoichiometry approaching, about or which is 1-2:1-2:10 (VP1:VP2:VP3).

In certain embodiments, AAV particles are produced in viral production cells (such as mammalian or insect cells) wherein all three VP proteins are expressed at a stoichiometry approaching, about or which is 2-3:0-3:10 (VP1:VP2:VP3).

In certain embodiments, AAV particles are produced in viral production cells (such as mammalian or insect cells) wherein all three VP proteins are expressed at a stoichiometry approaching, about or which is 2-3:2-3:10 (VP1:VP2:VP3).

In certain embodiments, AAV particles are produced in viral production cells (such as mammalian or insect cells) wherein all three VP proteins are expressed at a stoichiometry approaching, about or which is 3:3:10 (VP1:VP2:VP3).

In certain embodiments, AAV particles are produced in viral production cells (such as mammalian or insect cells) wherein all three VP proteins are expressed at a stoichiometry approaching, about or which is 3-5:0-5:10 (VP1:VP2:VP3).

In certain embodiments, AAV particles are produced in viral production cells (such as mammalian or insect cells) wherein all three VP proteins are expressed at a stoichiometry approaching, about or which is 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments of the present disclosure, Rep52 or Rep78 is transcribed from the baculoviral derived polyhedron promoter, (polh). Rep52 or Rep78 can also be transcribed from a weaker promoter, for example a deletion mutant of the IE-1 promoter, the AIE-1 promoter, has about 20% of the transcriptional activity of that IE-1 promoter. A promoter substantially homologous to the AIE-1 promoter may be used. In respect to promoters, a homology of at least 50%, 60%, 70%, 80%, 90% or more, is considered to be a substantially homologous promoter.

### Viral Production Cells and Vectors

### Mammalian Cells

Viral production of the present disclosure disclosed herein describes processes and methods for producing AAV particles or viral vector that contacts a target cell to deliver a payload construct, e.g. a recombinant AAV particle or viral construct, which includes a nucleotide encoding a payload molecule. The viral production cell may be selected from any biological organism, including prokaryotic (e.g., bacterial) cells, and eukaryotic cells, including, insect cells, yeast cells and mammalian cells.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO. W138, HeLa, HEK293, HEK293T (293T), Saos, C2C12, L cells, HT1080, HepG2 and primary fibroblast, hepatocyte and myoblast cells derived from mammals. Viral production cells can include cells derived from mammalian species including, but not limited to, human, monkey, mouse, rat, rabbit, and hamster or cell type, including but not limited to fibroblast, hepatocyte, tumor cell, cell line transformed cell, etc.

AAV viral production cells commonly used for production of recombinant AAV particles include, but is not limited to HEK293 cells, COS cells, C127, 3T3, CHO, HeLa cells, KB cells, BHK, and other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of each of which are herein incorporated by reference in their entireties insofar as they do no conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the contents of which are herein incorporated by reference in its entirety as related to the 293-10-3 packaging cell line and uses thereof.

In certain embodiments, of the present disclosure a cell line, such as a HeLA cell line, for trans-complementing E1 deleted adenoviral vectors, which encoding adenovirus Ela and adenovirus E1b under the control of a phosphoglycerate kinase (PGK) promoter can be used for AAV particle production as described in U.S. Pat. No. 6,365,394, the contents of which are incorporated herein by reference in their entirety as related to the HeLA cell line and uses thereof.

In certain embodiments, AAV particles are produced in mammalian cells using a triple transfection method wherein a payload construct, parvoviral Rep and parvoviral Cap and a helper construct are comprised within three different constructs. The triple transfection method of the three components of AAV particle production may be utilized to produce small lots of virus for assays including transduction efficiency, target tissue (tropism) evaluation, and stability.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other Ela trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the contents of which are incorporated by reference in its entirety as related to the production and processing of recombinant AAV virus particles. Production may also involve methods known in the art including those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethyleneimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can include optional medium changes (e.g. medium changes for cells in adhesion form, no medium changes for cells in suspension form, medium changes for cells in suspension form if desired). In certain embodiments, the transfection process can include transfection mediums such as DMEM or F17. In certain embodiments, the transfection medium can include serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80 C to 37 C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated by reference in their entireties as related to the measurement of particle concentrations).

### Insect Cells

Viral production of the present disclosure includes processes and methods for producing AAV particles or viral vectors that contact a target cell to deliver a payload construct, e.g. a recombinant viral construct, which includes a nucleotide encoding a payload molecule. In certain embodiments, the AAV particles or viral vectors of the present disclosure may be produced in a viral production cell that includes an insect cell.

Growing conditions for insect cells in culture, and production of heterologous products in insect cells in culture are well-known in the art, see U.S. Pat. No. 6,204,059, the contents of which are herein incorporated by reference in their entirety as related to the growth and use of insect cells in viral production.

Any insect cell which allows for replication of parvovirus and which can be maintained in culture can be used in accordance with the present disclosure. AAV viral production cells commonly used for production of recombinant AAV particles include, but is not limited to, Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O′Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88: 4646-50 (1991); Ruffing et al., J. Vir. 66:6922-30 (1992); Kimbauer et al.,Vir.219:37-44 (1996); Zhao et al., Vir.272:382-93 (2000); and Samulski et al., U.S. Pat. No. 6,204,059, the contents of each of which are herein incorporated by reference in their entirety as related to the use of insect cells in viral production.

In one embodiment, the AAV particles are made using the methods described in WO2015/191508, the contents of which are herein incorporated by reference in their entirety insofar as they do not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/ baculoviral system, which can be used for high levels of proteins, as described in U.S. Pat. No. 6,660,521, the contents of which are herein incorporated by reference in their entirety as related to the production of viral particles.

Expansion, culturing, transfection, infection and storage of insect cells can be carried out in any cell culture media, cell transfection media or storage media known in the art, including Hyclone SFX Insect Cell Culture Media, Expression System ESF AF Insect Cell Culture Medium, ThermoFisher Sf900II media, ThermoFisher Sf900III media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus-Production Systems

In certain embodiments, processes of the present disclosure can include production of AAV particles or viral vectors in a baculoviral system using a viral expression construct and a payload construct vector. In certain embodiments, the baculoviral system includes Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can include the viral expression construct (Expression BEV), and one or more group which can include the payload construct (Payload BEV). The baculoviruses may be used to infect a viral production cell for production of AAV particles or viral vector.

In certain embodiments, the process includes transfection of a single viral replication cell population to produce a single baculovirus (BEV) group which includes both the viral expression construct and the payload construct. These baculoviruses may be used to infect a viral production cell for production of AAV particles or viral vector.

In certain embodiments, BEVs are produced using a Bacmid Transfection agent, such as Promega FuGENE HD, WFI water, or ThermoFisher Cellfectin II Reagent. In certain embodiments, BEVs are produced and expanded in viral production cells, such as an insect cell.

In certain embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naive population of production cells. In certain embodiments, a bank of seed BIICs is stored at −80° C. or in LN₂ vapor.

Baculoviruses are made of several essential proteins which are essential for the function and replication of the Baculovirus, such as replication proteins, envelope proteins and capsid proteins. The Baculovirus genome thus includes several essential-gene nucleotide sequences encoding the essential proteins. As a non-limiting example, the genome can include an essential-gene region which includes an essential-gene nucleotide sequence encoding an essential protein for the Baculovirus construct. The essential protein can include: GP64 baculovirus envelope protein, VP39 baculovirus capsid protein, or other similar essential proteins for the Baculovirus construct.

Baculovirus expression vectors (BEV) for producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 Feb;80(4):1874-85, the contents of which are herein incorporated by reference in their entirety as related to the production and use of BEVs and viral particles.

Production of AAV particles with baculovirus in an insect cell system may address known baculovirus genetic and physical instability.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko DJ et al. Protein Expr Purif. 2009 June; 65(2):122-32, the contents of which are herein incorporated by reference in their entirety as related to the production and use of BEVs and viral particles.

A genetically stable baculovirus may be used to produce a source of the one or more of the components for producing AAV particles in invertebrate cells. In certain embodiments, defective baculovirus expression vectors may be maintained episomally in insect cells. In such an embodiment the corresponding bacmid vector is engineered with replication control elements, including but not limited to promoters, enhancers, and/or cell-cycle regulated replication elements.

In certain embodiments, baculoviruses may be engineered with a marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In certain embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In certain embodiments, the Baculovirus expression vectors (BEV) are based on the AcMNPV baculovirus or BmNPV baculovirus BmNPV. In certain embodiments, a bacmid of the present disclosure is based on (i.e. engineered variant of) an AcMNPV bacmid such as bmon14272, vAce25ko or vAclef11KO.

In certain embodiments, the Baculovirus expression vectors (BEV) is a BEV in which the baculoviral v-cath gene has been deleted (“v-cath deleted BEV”) or mutated.

Viral production bacmids of the present disclosure can include deletion of certain baculoviral genes or loci.

### Other

In certain embodiments expression hosts include, but are not limited to, bacterial species within the genera Escherichia, Bacillus, Pseudomonas, Salmonella.

In certain embodiments, a host cell which includes AAV rep and cap genes stably integrated within the cell's chromosomes, may be used for AAV particle production. In a non-limiting example, a host cell which has stably integrated in its chromosome at least two copies of an AAV rep gene and AAV cap gene may be used to produce the AAV particle according to the methods and constructs described in U.S. Pat. No. 7,238,526, the contents of which are incorporated herein by reference in their entirety as related to the production of viral particles.

In certain embodiments, the AAV particle can be produced in a host cell stably transformed with a molecule comprising the nucleic acid sequences which permit the regulated expression of a rare restriction enzyme in the host cell, as described in US20030092161 and EP1183380, the contents of which are herein incorporated by reference in their entirety as related to the production of viral particles.

In certain embodiments, production methods and cell lines to produce the AAV particle may include, but are not limited to those taught in PCT/US1996/010245, PCT/US1997/015716, PCT/US1997/015691, PCT/US1998/019479, PCT/US1998/019463, PCT/US2000/000415, PCT/US2000/040872, PCT/US2004/016614, PCT/US2007/010055, PCT/US1999/005870, PCT/US2000/004755, U.S. patent application Ser. No. 08/549489, U.S. Ser. No. 08/462,014, U.S. Ser. No. 09/659,203, U.S. Ser. No. 10/246,447, U.S. Pat. No. 10/465,302, U.S. Pat. Nos. 6,281,010, 6,270,996, 6,261,551, 5,756,283 (Assigned to NIH), U.S. Pat. Nos. 6,428,988, 6,274,354, 6,943,019, 6,482,634, (Assigned to NIH: U.S. Pat. Nos. 7,238,526, 6,475,769), U.S. Pat. No. 6,365,394 (Assigned to NIH), U.S. Pat. No. 7,491,508, 7,291,498, 7,022,519, 6,485,966, 6,953,690, 6,258,595, EP2018421, EP1064393, EP1163354, EP835321, EP931158, EP950111, EP1015619, EP1183380, EP2018421, EP1226264, EP1636370, EP1163354, EP1064393, US20030032613, US20020102714, US20030073232, US20030040101 (Assigned to NIH), US20060003451, US20020090717, US20030092161, US20070231303, US20060211115, US20090275107, US2007004042, US20030119191, US20020019050, the contents of each of which are incorporated herein by reference in their entirety insofar as they do no conflict with the present disclosure.

### Viral Production Systems

### Large-Scale Production

In certain embodiments, AAV particle production may be modified to increase the scale of production. Large scale viral production methods according to the present disclosure may include any of the processes or processing steps taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

Methods of increasing AAV particle production scale typically include increasing the number of viral production cells. In certain embodiments, viral production cells include adherent cells. To increase the scale of AAV particle production by adherent viral production cells, larger cell culture surfaces are required. In certain embodiments, large-scale production methods include the use of roller bottles to increase cell culture surfaces. Other cell culture substrates with increased surface areas are known in the art. Examples of additional adherent cell culture products with increased surface areas include, but are not limited to iCELLis (Pall Corp, Port Washington, N.Y.), CELLSTACK®, CELLCUBE® (Corning Corp., Corning, N.Y.) and NUNC™ CELL FACTORY™ (Thermo Scientific, Waltham, Mass.) In certain embodiments, large-scale adherent cell surfaces may include from about 1,000 cm² to about 100,000 cm².

In certain embodiments, large-scale viral production methods of the present disclosure may include the use of suspension cell cultures. Suspension cell culture can allow for significantly increased numbers of cells. Typically, the number of adherent cells that can be grown on about 10-50 cm² of surface area can be grown in about 1 cm³ volume in suspension.

In certain embodiments, large-scale cell cultures may include from about 10⁷ to about 10⁹ cells, from about 10⁸ to about 10¹⁰ cells, from about 10⁹ to about 10¹² cells or at least 10¹² cells. In certain embodiments, large-scale cultures may produce from about 10⁹ to about 10¹², from about 10¹⁰ to about 10¹³, from about 10¹¹ to about 10¹⁴, from about 10¹² to about 10¹⁵ or at least 10¹⁵ AAV particles.

Transfection of replication cells in large-scale culture formats may be carried out according to any methods known in the art. For large-scale adherent cell cultures, transfection methods may include, but are not limited to the use of inorganic compounds (e.g. calcium phosphate,) organic compounds (e.g. polyethyleneimine (PEI)) or the use of non-chemical methods (e.g. electroporation). With cells grown in suspension, transfection methods may include, but are not limited to the use of inorganic compounds (e.g. calcium phosphate,) organic compounds (e.g. polyethyleneimine (PEI)) or the use of non-chemical methods (e.g. electroporation). In certain embodiments, transfection of large-scale suspension cultures may be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes may be formed for introduction of plasmids to be transfected. In certain embodiments, cells being transfected with PEI-DNA complexes may be ‘shocked’ prior to transfection. This includes lowering cell culture temperatures to 4° C. for a period of about 1 hour. In certain embodiments, cell cultures may be shocked for a period of from about 10 minutes to about 5 hours. In certain embodiments, cell cultures may be shocked at a temperature of from about 0° C. to about 20° C.

In certain embodiments, transfections may include one or more vectors for expression of an RNA effector molecule to reduce expression of nucleic acids from one or more payload construct. Such methods may enhance the production of AAV particles by reducing cellular resources wasted on expressing payload constructs. In certain embodiments, such methods may be carried according to those taught in US Publication No. US2014/0099666, the contents of which are herein incorporated by reference in their entirety.

### Bioreactors

In certain embodiments, cell culture bioreactors may be used for large scale production of AAV particles. In certain embodiments, bioreactors include stirred tank reactors. Such reactors generally include a vessel, typically cylindrical in shape, with a stirrer (e.g. impeller.) In certain embodiments, such bioreactor vessels may be placed within a water jacket to control vessel temperature and/or to minimize effects from ambient temperature changes.

Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In certain embodiments, animal cell cultures may be maintained in bioreactors with rounded vessel bottoms.

In certain embodiments, bioreactor vessels may be warmed through the use of a thermocirculator. Thermocirculators pump heated water around water jackets. In certain embodiments, heated water may be pumped through pipes (e.g. coiled pipes) that are present within bioreactor vessels. In certain embodiments, warm air may be circulated around bioreactors, including, but not limited to air space directly above culture medium. Additionally, pH and CO2 levels may be maintained to optimize cell viability.

In certain embodiments, bioreactors may include hollow-fiber reactors. Hollow-fiber bioreactors may support the culture of both anchorage dependent and anchorage independent cells. Further bioreactors may include, but are not limited to, packed-bed or fixed-bed bioreactors. Such bioreactors may include vessels with glass beads for adherent cell attachment. Further packed-bed reactors may include ceramic beads.

In certain embodiments, viral particles are produced through the use of a disposable bioreactor. In certain embodiments, bioreactors may include GE WAVE bioreactor, a GE Xcellerax Bioreactor, a Sartorius Biostat Bioreactor, a ThermoFisher Hyclone Bioreactor, or a Pall Allegro Bioreactor.

In certain embodiments, AAV particle production in cell bioreactor cultures may be carried out according to the methods or systems taught in U.S. Pat. Nos. 5,064764, 6,194,191, 6,566,118, 8,137,948 or US Patent Application No. US2011/0229971, the contents of each of which are herein incorporated by reference in their entirety.

### Expansion of Viral Production Cell (VPC) Mixtures

In certain embodiments, an AAV particle or viral vector of the present disclosure may be produced in a viral production cell (VPC), such as an insect cell. Production cells can be sourced from a Cell Bank (CB) and are often stored in frozen cell banks.

In certain embodiments, a viral production cell from a Cell Bank is provided in frozen form. The vial of frozen cells is thawed, typically until ice crystal dissipate. In certain embodiments, the frozen cells are thawed at a temperature between 10-50° C., 15-40° C., 20-30 ° C., 25-50° C., 30-45° C., 35-40° C., or 37-39° C. In certain embodiments, the frozen viral production cells are thawed using a heated water bath.

In certain embodiments, a thawed CB cell mixture will have a cell density of 1.0×10⁴-1.0×10⁹ cells/mL. In certain embodiments, the thawed CB cell mixture has a cell density of 1.0×10⁴-2.5×10⁴ cells/mL, 2.5×10⁴-5.0×10⁴ cells/mL, 5.0×10⁴-7.5×10⁴ cells/mL, 7.5×10⁴-1.0×10⁵ cells/mL, 1.0×10⁵-2.5×10⁵ cells/mL, 2.5×10⁵-5.0×10⁵ cells/mL, 5.0×10⁵-7.5×10⁵ cells/mL, 7.5×10⁵-1.0×10⁶ cells/mL, 1.0×10⁶-2.5×10⁶ cells/mL, 2.5×10⁶-5.0×10⁶ cells/mL, 5.0×10⁶-7.5×10⁶ cells/mL, 7.5×10⁶-1.0×10⁷ cells/mL, 1.0×10⁷-2.5×10⁷ cells/mL, 2.5×10⁷-5.0×10⁷ cells/mL, 5.0×10⁷-7.5×10⁷ cells/mL, 7.5×10⁷-1.0×10⁸ cells/mL, 1.0×10⁸-2.5×10⁸ cells/mL, 2.5×10⁸-5.0×10⁸ cells/mL, 5.0×10⁸-7.5×10⁸ cells/mL, or 7.5×10⁸-1.0×10⁹ cells/mL.

In certain embodiments, the volume of the CB cell mixture is expanded. This process is commonly referred to as a Seed Train, Seed Expansion, or CB Cellular Expansion. Cellular/Seed expansion can include successive steps of seeding and expanding a cell mixture through multiple expansion steps using successively larger working volumes. In certain embodiments, cellular expansion can include one, two, three, four, five, six, seven, or more than seven expansion steps. In certain embodiments, the working volume in the cellular expansion can include one or more of the following working volumes or working volume ranges: 5 mL, 10 mL, 20 mL, 5-20 mL, 25 mL, 30 mL, 40 mL, 50 mL, 20-50 mL, 75 mL, 100 mL, 125 mL, 150 mL, 175 mL, 200 mL, 50-200 mL, 250 mL, 300 mL, 400 mL, 500 mL, 750 mL, 1000 mL, 250-1000 mL, 1250 mL, 1500 mL, 1750 mL, 2000 mL, 1000-2000 mL, 2250 mL, 2500 mL, 2750 mL, 3000 mL, 2000-3000 mL, 3500 mL, 4000 mL, 4500 mL, 5000 mL, 3000-5000 mL, 5.5 L, 6.0 L, 7.0 L, 8.0 L, 9.0 L, 10.0 L, and 5.0-10.0 L.

In certain embodiments, a volume of cells from a first expanded cell mixture can be used to seed a second, separate Seed Train/Seed Expansion (instead of using thawed CB cell mixture). This process is commonly referred to as rolling inoculum. In certain embodiments, rolling inoculum is used in a series of two or more (e.g. two, three, four or five) separate Seed Trains/Seed Expansions.

In certain embodiments, large-volume cellular expansion can include the use of a bioreactor, such as a GE WAVE bioreactor, a GE Xcellerax Bioreactor, a Sartorius Biostat Bioreactor, a ThermoFisher Hyclone Bioreactor, or a Pall Allegro Bioreactor.

In certain embodiments, the cell density within a working volume is expanded to a target output cell density. In certain embodiments, the output cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the output cell density of a working volume provides a seeding cell density for a larger, successive working volume. In certain embodiments, the seeding cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, cellular expansion can last for 1-50 days. Each cellular expansion step or the total cellular expansion can last for 1-10 days, 1-5 days, 1-3 days, 2-3 days, 2-4 days, 2-5 days, 2-6 days, 3-4 days, 3-5 days, 3-6 days, 3-8 days, 4-5 days, 4-6 days, 4-8 days, 5-6 days, or 5-8 days. In certain embodiments, each cellular expansion step or the total cellular expansion can last for 1-100 generations, 1-1000 generations, 100-1000 generation, 100 generations or more, or 1000 generation or more.

In certain embodiments, infected or transfected production cells can be expanded in the same manner as CB cell mixtures, as set forth in the present disclosure.

### Infection of Viral Production Cells

In certain embodiments, AAV particles of the present disclosure are produced in a viral production cell (VPC), such as an insect cell, by infecting the VPC with a viral vector which includes an AAV expression construct and/or a viral vector which includes an AAV payload construct. In certain embodiments, the VPC is infected with an Expression BEV which includes an AAV expression construct and a Payload BEV which includes an AAV payload construct.

In certain embodiments, AAV particles are produced by infecting a VPC with a viral vector which includes both an AAV expression construct and an AAV payload construct. In certain embodiments, the VPC is infected with a single BEV which includes both an AAV expression construct and an AAV payload construct.

In certain embodiments, VPCs (such as insect cells) are infected using Infection BIICs in an infection process which includes the following steps: (i) A collection of VPCs are seeded into a Production Bioreactor; (ii) The seeded VPCs can optionally be expanded to a target working volume and cell density; (iii) Infection BIICs which include Expression BEVs and Infection BIICs which include Payload BEVs are injected into the Production Bioreactor, resulting in infected viral production cells; and (iv) incubation of the infected viral production cells to produce AAV particles within the viral production cells.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, Infection BIICs are combined with the VPCs in target ratios of VPC-to-BIIC. In certain embodiments, the VPC-to-BIIC infection ratio (volume to volume) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ BIIC-per-VPC. In certain embodiments, the VPC-to-BIIC infection ratio (cell to cell) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ BIIC-per-VPC.

In certain embodiments, Infection BIICs which include Expression BEVs and Infection BIICs which include Payload BEVs are combined with the VPCs in target BIIC-to-BIIC ratios. In certain embodiments, the ratio of Expression (Rep/Cap) BIICs to Payload BIICs is 10:1, 9:1, 8:1, 7:1, 6:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:9, 1:10, 3.5-4.5:1, 3-4:1, 2.5-3.5:1, 2-3:1, 1.5-2.5:1, 1-2:1, 1-1.5:1, 1:1-1.5, 1:1-2, 1:1.5-2.5, 1:2-3, 1:2.5-3.5, 1:3-4, 1:3.5-4.5, 1:4-5, 1:4.5-5.5, 1:5-6, 1:5.5-6.5, 1:6-7, or 1:6.5-7.5.

### Cell Lysis

Cells of the present disclosure, including, but not limited to viral production cells, may be subjected to cell lysis according to any methods known in the art. Cell lysis may be carried out to obtain one or more agents (e.g. viral particles) present within any cells of the disclosure. In certain embodiments, a bulk harvest of AAV particles and viral production cells is subjected to cell lysis according to the present disclosure. As used herein, “cell lysis” refers to the breaking of the cell wall to release the intracellular contents.

In certain embodiments, cell lysis may be carried out according to any of the methods or systems presented in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Cell lysis methods and systems may be chemical or mechanical. Chemical cell lysis typically includes contacting one or more cells with one or more lysis agent. Mechanical lysis typically includes subjecting one or more cells to one or more lysis conditions and/or one or more lysis forces. Lysis can also be completed by allowing the cells to degrade after reaching ˜0% viability.

In certain embodiments, chemical lysis may be used to lyse cells. As used herein, the term “lysis agent” refers to any agent that may aid in the disruption of a cell. In certain embodiments, lysis agents are introduced in solutions, termed lysis solutions or lysis buffers. As used herein, the term “lysis solution” refers to a solution (typically aqueous) including one or more lysis agent. In addition to lysis agents, lysis solutions may include one or more buffering agents, solubilizing agents, surfactants, preservatives, cryoprotectants, enzymes, enzyme inhibitors and/or chelators. Lysis buffers are lysis solutions including one or more buffering agent. Additional components of lysis solutions may include one or more solubilizing agent. As used herein, the term “solubilizing agent” refers to a compound that enhances the solubility of one or more components of a solution and/or the solubility of one or more entities to which solutions are applied. In certain embodiments, solubilizing agents enhance protein solubility. In certain embodiments, solubilizing agents are selected based on their ability to enhance protein solubility while maintaining protein conformation and/or activity.

Exemplary lysis agents may include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In certain embodiments, lysis agents may be selected from lysis salts, amphoteric agents, cationic agents, ionic detergents and non-ionic detergents. Lysis salts may include, but are not limited to, sodium chloride (NaCl) and potassium chloride (KC1.) Further lysis salts may include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety.

In certain embodiments, cell lysates agents include amino acids such as arginine, or acidified amino acid mixtures such as arginine HCl.

Concentrations of salts may be increased or decreased to obtain an effective concentration for the rupture of cell membranes. Amphoteric agents, as referred to herein, are compounds capable of reacting as an acid or a base. Amphoteric agents may include, but are not limited to lysophosphatidylcholine, 3-((3-Cholamidopropyl) dimethylammonium)-1-propanesulfonate (CHAPS), ZWITTERGENT® and the like. Cationic agents may include, but are not limited to, cetyltrimethylammonium bromide (C (16) TAB) and Benzalkonium chloride. Lysis agents including detergents may include ionic detergents or non-ionic detergents.

Detergents may function to break apart or dissolve cell structures including, but not limited to cell membranes, cell walls, lipids, carbohydrates, lipoproteins and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. Some ionic detergents may include, but are not limited to, sodium dodecyl sulfate (SDS), cholate and deoxycholate. In certain embodiments, ionic detergents may be included in lysis solutions as a solubilizing agent. Non-ionic detergents may include, but are not limited to octylglucoside, digitonin, lubrol, C12E8, TWEEN®-20, TWEEN®-80, Triton X-100, Triton X-114, Brij-35, Brij-58, and Noniodet P-40. Non-ionic detergents are typically weaker lysis agents but may be included as solubilizing agents for solubilizing cellular and/or viral proteins. Further lysis agents may include enzymes and urea. In certain embodiments, one or more lysis agents may be combined in a lysis solution in order to enhance one or more of cell lysis and protein solubility. In certain embodiments, enzyme inhibitors may be included in lysis solutions in order to prevent proteolysis that may be triggered by cell membrane disruption.

In certain embodiments, cell lysates generated from adherent cell cultures may be treated with one more nuclease, such as Benzonase nuclease (Grade I, 99% pure) or c-LEcta Denarase nuclease (formerly Sartorius Denarase). In certain embodiments, nuclease is added to lower the viscosity of the lysates caused by liberated DNA.

In certain embodiments, chemical lysis uses a single chemical lysis mixture. In certain embodiments, chemical lysis uses several lysis agents added in series to provide a final chemical lysis mixture.

In certain embodiments, a chemical lysis mixture includes an acidified amino acid mixture (such as arginine HC1), a non-ionic detergent (such as Triton X-100), and a nuclease (such as Benzonase nuclease). In certain embodiments, the chemical lysis mixture can include an acid or base to provide a target lysis pH. In certain embodiments, the lysis pH is between 6.0-7.0, 6.5-7.0, 6.5-7.5, or 7.0-7.5.

In certain embodiments, mechanical cell lysis is carried out. Mechanical cell lysis methods may include the use of one or more lysis condition and/or one or more lysis force. As used herein, the term “lysis condition” refers to a state or circumstance that promotes cellular disruption. Lysis conditions may include certain temperatures, pressures, osmotic purity, salinity and the like. In certain embodiments, lysis conditions include increased or decreased temperatures. According to certain embodiments, lysis conditions include changes in temperature to promote cellular disruption. Cell lysis carried out according to such embodiments may include freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further include one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In certain embodiments, one or more cryoprotectants are included in cell solutions undergoing freeze-thaw lysis. As used herein, the term “cryoprotectant” refers to an agent used to protect one or more substance from damage due to freezing. Cryoprotectants may include any of those taught in US Publication No. US2013/0323302 or U.S. Pat. Nos. 6,503,888, 6,180,613, 7,888,096, 7,091,030, the contents of each of which are herein incorporated by reference in their entirety. In certain embodiments, cryoprotectants may include, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In certain embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may include, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may include high shear fluid forces. According to such methods of mechanical lysis, a microfluidizer may be used. Microfluidizers typically include an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g. viral particles.) Other mechanical lysis methods may include physical disruption of cells by scraping.

Cell lysis methods may be selected based on the cell culture format of cells to be lysed. For example, with adherent cell cultures, some chemical and mechanical lysis methods may be used. Such mechanical lysis methods may include freeze-thaw lysis or scraping. In another example, chemical lysis of adherent cell cultures may be carried out through incubation with lysis solutions including surfactant, such as Triton-X-100.

In certain embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification and Purification: General

Cell lysates including viral particles may be subjected to clarification and purification. Clarification generally refers to the initial steps taken in the purification of viral particles from cell lysates and serves to prepare lysates for further purification by removing larger, insoluble debris from a bulk lysis harvest. Viral production can include clarification steps at any point in the viral production process. Clarification steps may include, but are not limited to, centrifugation and filtration. During clarification, centrifugation may be carried out at low speeds to remove larger debris only. Similarly, filtration may be carried out using filters with larger pore sizes so that only larger debris is removed.

Purification generally refers to the final steps taken in the purification and concentration of viral particles from cell lysates by removing smaller debris from a clarified lysis harvest in preparing a final Pooled Drug Substance. Viral production can include purification steps at any point in the viral production process. Purification steps may include, but are not limited to, filtration and chromatography. Filtration may be carried out using filters with smaller pore sizes to remove smaller debris from the product or with larger pore sizes to retain larger debris from the product. Filtration may be used to alter the concentration and/or contents of a viral production pool or stream. Chromatography may be carried out to selectively separate target particles from a pool of impurities.

Large scale production of high-concentration AAV formulations is complicated by the tendency for high concentrations of AAV particles to aggregate or agglomerate. Small scale clarification and concentration systems, such as dialysis cassettes or spin centrifugation, are generally not sufficiently scalable for large-scale production. The present disclosure provides embodiments of a clarification, purification and concentration system for processing large volumes of high-concentration AAV production formulations. In certain embodiments, the large-volume clarification system includes one or more of the following processing steps: Depth Filtration, Microfiltration (e.g. 0.2 μm Filtration), Affinity Chromatography, Ion Exchange Chromatography such as anion exchange chromatography (AEX) or cation exchange chromatography (CEX), a tangential flow filtration system (TFF), Nanofiltration (e.g. Virus Retentive Filtration (VRF)), Final Filtration (FF), and Fill Filtration.

Objectives of viral clarification and purification include high throughput processing of cell lysates and to optimize ultimate viral recovery. Advantages of including clarification and purification steps of the present disclosure include scalability for processing of larger volumes of lysate. In certain embodiments, clarification and purification may be carried out according to any of the methods or systems presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

In certain embodiments, the compositions including at least one AAV particle may be isolated or purified using the methods or systems described in U.S. Pat. Nos. 6,146,874, 6,660,514, 8,283,151 or 8,524,446, the contents of which are herein incorporated by reference in their entirety.

### Clarification and Purification: Centrifugation

According to certain embodiments, cell lysates may be clarified by one or more centrifugation steps. Centrifugation may be used to pellet insoluble particles in the lysate. During clarification, centrifugation strength (which can be expressed in terms of gravitational units (g), which represents multiples of standard gravitational force) may be lower than in subsequent purification steps. In certain embodiments, centrifugation may be carried out on cell lysates at a gravitation force from about 200 g to about 800 g, from about 500 g to about 1500 g, from about 1000 g to about 5000 g, from about 1200 g to about 10000 g or from about 8000 g to about 15000 g. In certain embodiments, cell lysate centrifugation is carried out at 8000 g for 15 minutes. In certain embodiments, density gradient centrifugation may be carried out in order to partition particulates in the cell lysate by sedimentation rate. Gradients used according to methods or systems of the present disclosure may include, but are not limited to, cesium chloride gradients and iodixanol step gradients. In certain embodiments, centrifugation uses a decanter centrifuge system. In certain embodiments, centrifugation uses a disc-stack centrifuge system. In certain embodiments, centrifugation includes ultracentrifugation, such two-cycle CsCl gradient ultracentrifugation or iodixanol discontinuous density gradient ultracentrifugation.

### Clarification and Purification: Filtration

In certain embodiments, one or more microfiltration, nanofiltration and/or ultrafiltration steps may be used during clarification, purification and/or sterilization. The one or more microfiltration, nanofiltration or ultrafiltration steps can include the use of a filtration system such as EMD Millipore Express SHC XL10 0.5/0.2 μm filter, EMD Millipore Express SHCXL6000 0.5/0.2 μm filter, EMD Millipore Express SHCXL150 filter, EMD Millipore Millipak Gamma Gold 0.22 μm filter (dual-in-line sterilizing grade filters), a Pall Supor EKV, 0.2 μm sterilizing-grade filter, Asahi Planova 35N, Millipore Viresolve NFR or a Sartorius Sartopore 2XLG, 0.8/0.2 μm.

In certain embodiments, one or more microfiltration steps may be used during clarification, purification and/or sterilization. Microfiltration utilizes microfiltration membranes with pore sizes typically between 0.1 μm and 10 μm. Microfiltration is generally used for general clarification, sterilization, and removal of microparticulates. In certain embodiments, microfiltration is used to remove aggregated clumps of viral particles. In certain embodiments, a production process or system of the present disclosure includes at least one microfiltration step. The one or more microfiltration steps can include a Depth Filtration step with a Depth Filtration system, such as EMD Millipore Millistak⁺ POD filter (DOHC media series) or Sartorius Sartopore filter series. Microfiltration systems of the present disclosure can be pre-rinsed, packed, equilibrated, flushed, processed, eluted, washed or cleaned with formulations known to those in the art, including AAV pharmaceutical, processing and storage formulations of the present disclosure.

In certain embodiments, one or more ultrafiltration steps may be used during clarification and purification. The ultrafiltration steps can be used for concentrating, formulating, desalting or dehydrating either processing and/or formulation solutions of the present disclosure. Ultrafiltration utilizes ultrafiltration membranes, with pore sizes typically between 0.001 and 0.1 μm. Ultrafiltration membranes can also be defined by their molecular weight cutoff (MWCO) and can have a range from 1 kD to 500 kD. Ultrafiltration is generally used for concentrating and formulating dissolved biomolecules such as proteins, peptides, plasmids, viral particles, nucleic acids, and carbohydrates. Ultrafiltration systems of the present disclosure can be pre-rinsed, packed, equilibrated, flushed, processed, eluted, washed or cleaned with formulations known to those in the art, including AAV pharmaceutical, processing and storage formulations of the present disclosure.

In certain embodiments, one or more nanofiltration steps may be used during clarification and purification. Nanofiltration utilizes nanofiltration membranes, with pore sizes typically less than 100 nm. Nanofiltration is generally used for removal of unwanted endogenous viral impurities (e.g. baculovirus). In certain embodiments, nanofiltration can include viral removal filtration (VRF). VRF filters can have a filtration size typically between 15 nm and 100 nm. Examples of VRF filters include (but are not limited to): Planova 15N, Planova 20N, and Planova 35N (Asahi-Kasei Corp, Tokyo, Japan); and Viresolve NFP and Viresolve NFR (Millipore Corp, Billerica, Mass., USA). Nanofiltration systems of the present disclosure can be pre-rinsed, packed, equilibrated, flushed, processed, eluted, washed or cleaned with formulations known to those in the art, including AAV pharmaceutical, processing and storage formulations of the present disclosure. In certain embodiments, nanofiltration is used to remove aggregated clumps of viral particles.

In certain embodiments, one or more tangential flow filtration (TFF) (also known as cross-flow filtration) steps may be used during clarification and purification. Tangential flow filtration is a form of membrane filtration in which a feed stream (which includes the target agent/particle to be clarified and concentrated) flows from a feed tank into a filtration module or cartridge. Within the TFF filtration module, the feed stream passes parallel to a membrane surface, such that one portion of the stream passes through the membrane (permeate/filtrate) while the remainder of the stream (retentate) is recirculated back through the filtration system and into the feed tank.

In certain embodiments, the TFF filtration module can be a flat plate module (stacked planar cassette), a spiral wound module (spiral-wound membrane layers), or a hollow fiber module (bundle of membrane tubes). Examples of TFF systems for use in the present disclosure include, but are not limited to: Spectrum mPES Hollow Fiber TFF system (0.5 mm fiber ID, 100 kDA MWCO) or Millipore Ultracel PLCTK system with Pellicon-3 cassette (0.57 m², 30 kDA MWCO).

New buffer materials can be added to the TFF feed tank as the feed stream is circulated through the TFF filtration system. In certain embodiments, buffer materials can be fully replenished as the flow stream circulates through the TFF filtration system. In this embodiment, buffer material is added to the stream in equal amounts to the buffer material lost in the permeate, resulting in a constant concentration. In certain embodiments, buffer materials can be reduced as the flow stream circulates through the filtration system. In this embodiment, a reduced amount of buffer material is added to the stream relative to the buffer material lost in the permeate, resulting in an increased concentration. In certain embodiments, buffer materials can be replaced as the flow stream circulates through the filtration system. In this embodiment, the buffer added to stream is different from buffer materials lost in the permeate, resulting in an eventual replacement of buffer material in the stream. TFF systems of the present disclosure can be pre-rinsed, packed, equilibrated, flushed, processed, eluted, washed or cleaned with formulations known to those in the art, including AAV pharmaceutical, processing and storage formulations of the present disclosure.

In certain embodiments, a TFF load pool can be spiked with an excipient or diluent prior to filtration. In certain embodiments, a TFF load pool is spiked with a high-salt mixture (such as sodium chloride or potassium chloride) prior to filtration. In certain embodiments, a TFF load pool is spiked with a high-sugar mixture (such as 50% w/v sucrose) prior to filtration.

The effectiveness of TFF processing can depend on several factors, including (but not limited to): shear stress from flow design, cross-flow rate, filtrate flow control, transmembrane pressure (TMP), membrane conditioning, membrane composition (e.g. hollow fiber construction) and design (e.g. surface area), system flow design, reservoir design, and mixing strategy. In certain embodiments, the filtration membrane can be exposed to pre-TFF membrane conditioning.

In certain embodiments, TFF processing can include one or more microfiltration stages. In certain embodiments, TFF processing can include one or more ultrafiltration stages. In certain embodiments, TFF processing can include one or more nanofiltration stages.

In certain embodiments, TFF processing can include one or more concentration stages, such as an ultrafiltration (UF) or microfiltration (MF) concentration stage. In the concentration stage, a reduced amount of buffer material is replaced as the stream circulates through the filtration system (relative to the amount of buffer material lost as permeate). The failure to completely replace all of the buffer material lost in the permeate results in an increased concentration of viral particles within the filtration stream. In certain embodiments, an increased amount of buffer material is replaced as the stream circulates through the filtration system. The incorporation of excess buffer material relative to the amount of buffer material lost in the permeate results in a decreased concentration of viral particles within the filtration stream.

In certain embodiments, TFF processing can include one or more diafiltration (DF) stages. The diafiltration stage includes replacement of a first buffer material (such as a high salt material) within a second buffer material (such a low-salt or zero-salt material). In this embodiment, a second buffer is added to flow stream which is different from a first buffer material lost in the permeate, resulting in an eventual replacement of buffer material in the stream.

In certain embodiments, TFF processing can include multiple stages in series. In certain embodiments, a TFF processing process can include an ultrafiltration (UF) concentration stage followed by a diafiltration stage (DF). In certain embodiments, a TFF processing can include a diafiltration stage followed by an ultrafiltration concentration stage. In certain embodiments, a TFF processing can include a first diafiltration stage, followed by an ultrafiltration concentration stage, followed by a second diafiltration stage. In certain embodiments, a TFF processing can include a first diafiltration stage which incorporates a high-salt-low-sugar buffer material into the flow stream, followed by an ultrafiltration/concentration stage which results in a high concentration of the viral material in the flow stream, followed by a second diafiltration stage which incorporates a low-salt-high-sugar or zero-salt-high-sugar buffer material into the flow stream. In certain embodiments, the salt can be sodium chloride, sodium phosphate, potassium chloride, potassium phosphate, or a combination thereof. In certain embodiments, the sugar can be sucrose, such as a 5% w/v sucrose mixture or a 7% w/v sucrose mixture.

In certain embodiments, TFF processing can include multiple stages which occur contemporaneously. As a non-limiting example, a TFF clarification process can include an ultrafiltration stage which occurs contemporaneously with a concentration stage.

Methods of cell lysate clarification and purification by filtration are well understood in the art and may be carried out according to a variety of available methods including, but not limited to passive filtration and flow filtration. Filters used may include a variety of materials and pore sizes. For example, cell lysate filters may include pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In certain embodiments, clarification may include filtration through a filter with 2.0 μM pore size to remove large debris, followed by passage through a filter with 0.45 μM pore size to remove intact cells.

Filter materials may be composed of a variety of materials. Such materials may include, but are not limited to, polymeric materials and metal materials (e.g. sintered metal and pored aluminum.) Exemplary materials may include, but are not limited to nylon, cellulose materials (e.g. cellulose acetate), polyvinylidene fluoride (PVDF), polyethersulfone, polyamide, polysulfone, polypropylene, and polyethylene terephthalate. In certain embodiments, filters useful for clarification of cell lysates may include, but are not limited to ULTIPLEAT PROFILE™ filters (Pall Corporation, Port Washington, N.Y.), SUPOR™ membrane filters (Pall Corporation, Port Washington, N.Y.)

In certain embodiments, flow filtration may be carried out to increase filtration speed and/or effectiveness. In certain embodiments, flow filtration may include vacuum filtration. According to such methods, a vacuum is created on the side of the filter opposite that of cell lysate to be filtered. In certain embodiments, cell lysates may be passed through filters by centrifugal forces. In certain embodiments, a pump is used to force cell lysate through clarification filters. Flow rate of cell lysate through one or more filters may be modulated by adjusting one of channel size and/or fluid pressure.

### Clarification and Purification: Chromatography

In certain embodiments, AAV particles in a formulation may be clarified and purified from cell lysates through one or more chromatography steps using one or more different methods of chromatography. Chromatography refers to any number of methods known in the art for selectively separating out one or more elements from a mixture. Such methods may include, but are not limited to, ion exchange chromatography (e.g. cation exchange chromatography and anion exchange chromatography), affinity chromatography (e.g. immunoaffinity chromatography, metal affinity chromatography, pseudo affinity chromatography such as Blue Sepharose resins), hydrophobic interaction chromatography, size-exclusion chromatography, and multimodal chromatography (chromatographic methods that utilize more than one form of interaction between the stationary phase and analytes). In certain embodiments, methods or systems of viral chromatography may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Chromatography systems of the present disclosure can be pre-rinsed, packed, equilibrated, flushed, processed, eluted, washed or cleaned with formulations known to those in the art, including AAV pharmaceutical, processing and storage formulations of the present disclosure.

In certain embodiments, one or more ion exchange (IEX) chromatography steps may be used to isolate viral particles. The ion exchange step can include anion exchange (AEX) chromatography, cation exchange (CEX) chromatography, or a combination thereof. In certain embodiments, ion exchange chromatography is used in a bind/elute mode. Bind/elute IEX can be used by binding viral particles to a stationary phase based on charge-charge interactions between capsid proteins (or other charged components) of the viral particles and charged sites present on the stationary phase. This process can include the use of a column through which viral preparations (e.g. clarified lysates) are passed. After application of viral preparations to the charged stationary phase (e.g. column), bound viral particles may then be eluted from the stationary phase by applying an elution solution to disrupt the charge-charge interactions. Elution solutions may be optimized by adjusting salt concentration and/or pH to enhance recovery of bound viral particles. Depending on the charge of viral capsids being isolated, cation or anion exchange chromatography methods may be selected. In certain embodiments, ion exchange chromatography is used in a flow-through mode. Flow-through IEX can be used by binding non-viral impurities or unwanted viral particles to a stationary phase (based on charge-charge interactions) and allowing the target viral particles in the viral preparation to “flow through” the IEX system into a collection pool.

Methods or systems of ion exchange chromatography may include, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety. In certain embodiments, the IEX process uses an AEX chromatography system such as a Sartorius Sartobind Q membrane, a Millipore Fractogel TMAE HiCap(m) Flow-Through membrane, a GE Q Sepharose HP membrane, and Porox XQ. In certain embodiments, the IEX process uses a CEX system such as a Poros XS membrane.

In certain embodiments, one or more affinity chromatography steps, such as immunoaffinity chromatography, may be used to isolate viral particles. Immunoaffinity chromatography is a form of chromatography that utilizes one or more immune compounds (e.g. antibodies or antibody-related structures) to retain viral particles. Immune compounds may bind specifically to one or more structures on viral particle surfaces, including, but not limited to one or more viral coat protein. In certain embodiments, immune compounds may be specific for a particular viral variant. In certain embodiments, immune compounds may bind to multiple viral variants. In certain embodiments, immune compounds may include recombinant single-chain antibodies. Such recombinant single chain antibodies may include those described in Smith, R. H. et al., 2009. Mol. Ther. 17(11):1888-96, the contents of which are herein incorporated by reference in their entirety. Such immune compounds are capable of binding to several AAV capsid variants, including, but not limited to AAV1, AAV2, AAV6 and AAV8 or any of those taught herein. In certain embodiments, the AFC process uses a GE AVB Sepharose HP column resin, Poros AAV8 resins (ThermoFisher), Poros AAV9 resins (ThermoFisher) and Poros AAVX resins (ThermoFisher).

In certain embodiments, one or more size-exclusion chromatography (SEC) steps may be used to isolate viral particles. SEC may include the use of a gel to separate particles according to size. In viral particle purification, SEC filtration is sometimes referred to as “polishing.” In certain embodiments, SEC may be carried out to generate a final product that is near-homogenous. Such final products may in certain embodiments be used in pre-clinical studies and/or clinical studies (Kotin, R.M. 2011. Human Molecular Genetics. 20(1):R2-R6, the contents of which are herein incorporated by reference in their entirety.) In certain embodiments, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

### Measurement and Analysis

Expression of payloads or the downregulating effect of such payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

The viral genome titer is a starting point for many analytical and biological assays measuring quality attributes of an AAV product. Variability in viral genome titers can carry over into all the other downstream assays, with the potential to create a systematic bias. The current industry standard for measuring viral AAV genome titers is real-time PCR with quantitation through use of a calibration or standard curve (qPCR). qPCR has been shown to have a large dynamic range (5-6 logs), to have high sample throughput, and to have good intra-assay precision (based on intra-assay standardization). However, the nature of using a separate standard curves for separate sample batches introduces systematic variability. Inter-assay variability observed for qPCR can range from below 10% to greater than 30%. Additionally, site-to-site variability can be greater than 50%, which will greatly affect quantitation accuracy. There is a need for improved quantification methods which provide absolute copy numbers, thereby removing the systemic bias of qPCR calibration curves and corresponding decreased accuracy in downstream assays such as titer assays.

On possible alternative or addition to qPCR analysis is Droplet digital PCR (ddPCR). ddPCR provides good inter-assay precision (provides absolute quantificiation independent of standard curves) and corresponding improved accuracy in downstream titer assays. However, ddPCR assays can have low dynamic ranges, low sample throughput, and can be prone to errors due to high sensitivity. Furthermore, ddPCR is a newly developed quantification method and few studies have been completed to validate the use of ddPCR in certain applications (such as AAV titer analysis), or to compare the effectiveness of ddPCR relative to existing methods such as qPCR.

### Viral Vector Titer

The present disclosure presents methods and systems for measuring and analyzing the viral vector titer of a sample. In certain embodiments, accurate, precise and reproducible titers can be used to ensure more accurate, precise and reproducible potency readouts and can further be used to ensure confidence in experimental or clinical dosing. Titer and potency data, when combined, may serve to further inform one of skill in the art to viral vector characteristics and proper dosing parameters for enhanced efficacy and safety. Multiple measurements of titer and potency may further increase accuracy and precision in lot to lot comparisons and long-term stability studies.

Titer of a viral vector composition may be assessed by any means known in the art, including, but not limited to, quantitative polymerase chain reaction (qPCR) or droplet digital polymerase chain reaction (ddPCR). Studies comparing these methods for assessment of viral vector titer have been previously described (e.g., Lock et al., Hum. Gene Ther. Meth., 2014, 25:115-125, the contents of which are herein incorporated by reference in their entirety). In one embodiment, viral vector titer is assessed by qPCR. In one embodiment, viral vector titer is assessed by ddPCR. In one embodiment, viral vector titer is compared to a reference standard. In one embodiment, the viral vector titer of a reference standard is assessed by ddPCR, and the viral vector titer of a sample is assessed by qPCR.

The current industry standard, and well known in the art, qPCR is a real-time amplification method reliant on the use of fluorescent probes that hybridize to a specific DNA sequence. The qPCR assay consists of iterative cycles of denaturing, annealing and extension, and the number of cycles may be adjusted based on experimental needs. The fluorescence is monitored during the reaction and can be plotted as a set of standard amplification curves. These curves, when used in conjunction with set threshold values can be used to determine a concentration. The use of a separate standard curve can introduce systematic variability into the results.

ddPCR is a more recently developed PCR technique that allows for direct quantification of absolute DNA copy numbers, without the need for a standard curve. Water-oil emulsion droplets are used in an end point assay wherein each droplet undergoes an individual chemical reaction and has a separate readout. Absolute copy number can be quantified based on the ratio of positive to negative droplets of the unknown sample and this can be used to generate a raw concentration value. Quantification by ddPCR does not rely on a standard curve as necessary in the qPCR method.

While not wishing to be bound by theory, both qPCR and ddPCR methods have distinct advantages and/or disadvantages for their use in measurement of viral titer. qPCR is considered to provide a greater data range, increased sample throughput and increased intra-assay precision, while ddPCR is considered to demonstrate greater titer accuracy, without dependence on a standard curve and increased inter-assay precision and accuracy. The reliance on a standard curve for qPCR assessment is often considered a disadvantage, and the method is thought to have decreased titer accuracy and sensitivity. However, ddPCR is not without disadvantages as well, since the method is often considered to have decreased dynamic range, lower sample throughput and higher sensitivity which can magnify errors.

As variability can occur between readings, more than one batch of viral vector may be assayed by qPCR or ddPCR. One, two, three, four, five, six, seven, eight, nine, ten, or more than ten batches may be assayed. Additionally, the batches may be assessed for operator variability.

Increased precision and accuracy (inter- and intra-assay) of viral vector quantification by qPCR or ddPCR can result in decreased downstream variability in potency testing. Multiple measurements of potency can be used for further increasing precision of the potency readout.

In certain embodiments, the present disclosure provides methods of measuring viral vector titer. In certain embodiments, the method includes: providing a formulation which includes a collection of AAV vector particles produced using a set of viral expression constructs and payload constructs; and measuring/determing the viral titer of AAV vector particles in the formulation using ddPCR. In certain embodiments, the viral expression constructs and payload constructs are Baculovirus expression vectors (BEVs) within baculovirus infected insect cells (BIICs) from a bank of seed BIICs. In certain embodiments, the AAV vector particles are produced in a bioreactor vessel having a volume of about 50 L or less. In certain embodiments, the AAV vector particles are produced in a bioreactor vessel having a volume of about 100 L or more. In certain embodiments, the concentration of AAV vector particles in the formulation is adjusted to a target concentration, thereby providing a transducing formulation with a target multiplicity of infection (MOI) of AAV vector particles.

In certain embodiments, the method includes: providing a formulation which includes a collection of AAV vector particles produced using a set of viral expression constructs and payload constructs; and measuring/determing the viral titer of AAV vector particles in the formulation using qPCR. In certain embodiments, the viral expression constructs and payload constructs are Baculovirus expression vectors (BEVs) within baculovirus infected insect cells (BIICs) from a bank of seed BIICs. In certain embodiments, the AAV vector particles are produced in a bioreactor vessel having a volume of about 50 L or less. In certain embodiments, the AAV vector particles are produced in a bioreactor vessel having a volume of about 100 L or more. In certain embodiments, the concentration of AAV vector particles in the formulation is adjusted to a target concentration, thereby providing a transducing formulation with a target multiplicity of infection (MOI) of AAV vector particles.

### Viral Vector Potency

In certain embodiments, the present disclosure provides methods of measuring viral vector potency of AAV vector particles. In some embodiments, the viral vector comprises a polynucleotide encoding a molecule of interest (i.e. payload). In some embodiments, the present application provides methods to measure a viral vector's potency, wherein the viral vector comprises a polynucleotide encoding a molecule of interest, and potency is based on the activity of the molecule of interest. In some embodiments, the present application provides methods to measure a viral vector's potency, wherein the viral vector comprises a polynucleotide encoding a RNAi molecule of interest, and the potency is based on the knockdown and/or silencing activity of the RNAi molecule of interest.

In some embodiments, cells (such as HT1080 cells) are plated at a density of about 1×10² cells/well to about 1×10⁸ cells/well, about 1×10² cells/well to about 1×10⁷ cells/well, about 1×10² cells/well to about 1×10⁶ cells/well, about 1×10² cells/well to about 1×10⁵ cells/well, about 1×10² cells/well to about 1×10⁴ cells/well, about 1×10² cells/well to about 1×10³ cells/well, about 1×10³ cells/well to about 1×10⁴ cells/well, about 1×10³ cells/well to about 1×10⁵ cells/well, about 1×10³ cells/well to about 1×10⁶ cells/well, about 1×10⁴ cells/well to about 1×10⁶ cells/well, or about 1×10⁵ cells/well to about 1×10⁷ cells/well. In some embodiments, the HT1080 cells are plated at a density of about 1×10⁴ cells/well.

In some embodiments, cells are transduced with a viral vector, for example, AAV vector, encoding a protein of interest, lysed, and the cell lysate is collected. In some embodiments, cells are transduced with a viral vector, for example, AAV vector, encoding a protein of interest and harvested after about 18 to about 72 hours post transduction. In some embodiments, the cells are harvested after 24 hours post transduction. In some embodiments, the cells are harvested after about 24 to about 44 hours post transduction. In some embodiments, the cells are harvested after about 44 to about 52 hours post transduction. In some embodiments, the cells are harvested after about 52 to about 72 hours post transduction.

In some embodiments, the cells are lysed using chemical and/or mechanical lysis. In some embodiments, the chemical lysis comprises a lysis buffer comprising a protease inhibitor, phosphate buffered saline and Triton X100. In some embodiments, the cells can be frozen after the addition of the lysis buffer at -80° C. for about 30 min to about 72 hours. In some embodiments, the cells are centrifuged and cell lysates are collected. In some embodiments, this is performed by spinning the cells in a centrifuge at 3,750 RPM for 10 minutes at room temperature.

In some embodiments, cells are transduced with a viral vector at a certain multiplicity of infection (MOI) of AAV vector particles within a transducing formulation. In certain embodiments, the viral titer of AAV vector particles in the transducing formulation is calculated using qPCR. In certain embodiments, the viral titer of AAV vector particles in the transducing formulation is calculated using ddPCR. In certain embodiments, the viral titer of AAV vector particles in the transducing formulation is calculated using a combination of qPCR and ddPCR.

In certain embodiments, the present disclosure provides methods of measuring viral vector potency, including: providing a first transducing formulation which includes a first collection of AAV vector particles produced using a first set of viral production constructs (i.e. viral expression constructs and payload constructs); measuring/determing the viral titer of AAV vector particles in the first transducing formulation using ddPCR; optionally adjusting the concentration of AAV vector particles in the first transducing formulation; and measuring/determing the viral vector potency of the first collection of AAV vector particles in the first transducing formulation using an AAV potency assay. In certain embodiments, the method further includes: providing a second transducing formulation which includes a second collection of AAV vector particles produced using the first set of viral production constructs; measuring/determing the viral titer of AAV vector particles in the second transducing formulation using qPCR; optionally adjusting the concentration of AAV vector particles in the second transducing formulation; and measuring/determing the viral vector potency of the second collection of AAV vector particles in the second transducing formulation using an AAV potency assay. In certain embodiments, the method further includes comparing the viral vector potency of the second collection of AAV vector particles with the viral vector potency of the first collection of AAV vector particles. In certain embodiments, the method further includes suitably aliquoting the AAV formulation into a formulation container after the viral vector potency has been measured. In certain embodiments, the first set of viral production constructs (i.e. viral expression constructs and payload constructs) are Baculovirus expression vectors (BEVs) within baculovirus infected insect cells (BIICs) from a bank of seed BIICs. In certain embodiments, the first collection of AAV vector particles are produced in a bioreactor vessel having a volume of about 50 L or less. In certain embodiments, the second collection of AAV vector particles are produced in a bioreactor vessel having a volume of about 100 L or more. In certain embodiments, the viral vector potency of the first collection of AAV vector particles is a positive control for a defined acceptable range of viral vector potency for the second collection of AAV vector particles.

In certain embodiments, the present disclosure provides methods of measuring viral vector potency, including: providing a first transducing formulation which includes a first collection of AAV vector particles produced using a first set of viral production constructs (i.e. viral expression constructs and payload constructs); measuring/determing the viral titer of AAV vector particles in the first transducing formulation using qPCR; optionally adjusting the concentration of AAV vector particles in the first transducing formulation; and measuring/determing the viral vector potency of the first collection of AAV vector particles in the first transducing formulation using an AAV potency assay. In certain embodiments, the method further includes: providing a second transducing formulation which includes a second collection of AAV vector particles produced using first set of viral production constructs (i.e. viral expression constructs and payload constructs); measuring/determing the viral titer of AAV vector particles in the second transducing formulation using ddPCR; optionally adjusting the concentration of AAV vector particles in the second transducing formulation; and measuring/determing the viral vector potency of the second collection of AAV vector particles in the second transducing formulation using an AAV potency assay. In certain embodiments, the method further includes comparing the viral vector potency of the second collection of AAV vector particles with the viral vector potency of the first collection of AAV vector particles. In certain embodiments, the method further includes suitably aliquoting the AAV formulation into a formulation container after the viral vector potency has been measured. In certain embodiments, the first set of viral production constructs (i.e. viral expression constructs and payload constructs) are Baculovirus expression vectors (BEVs) within baculovirus infected insect cells (BIICs) from a bank of seed BIICs. In certain embodiments, the first collection of AAV vector particles are produced in a bioreactor vessel having a volume of about 50 L or less. In certain embodiments, the second collection of AAV vector particles are produced in a bioreactor vessel having a volume of about 100 L or more. In certain embodiments, the viral vector potency of the first collection of AAV vector particles is a positive control for a defined acceptable range of viral vector potency for the second collection of AAV vector particles.

In certain embodiments, the methods described herein can further comprise use of a positive control (i.e. viral vector reference standard) comprising a viral vector lot monitored for values within a defined acceptable range. If an assay run results in the values from the positive control that are outside the acceptable range, the assay run can be declared invalid. Such a positive control can provide benefit as a validity or acceptance criterion.

In certain embodiments, the step of measuring/determing the viral vector potency of a collection of AAV vector particles in a formulation using an AAV potency assay includes: determining a multiplicity of infection (MOI) for the AAV vector particles based on the titer (such as titer determined by qPCR, ddPCR or a combination thereof); transducing the AAV vector particles from the formulation into a target cell using the determined MOI, and under conditions in which the target cell will produce the payload molecule; lysing the target cells and collecting the resulting cell lysate sample; adding a molecule of interest to the cell lysate sample, wherein the molecule of interest interacts with the payload molecule to produce a product molecule; and measuring the amount of product molecule produced in the cell lysate, such that the potency of the AAV vector particle is measured. In certain embodiments, the method includes comparing the amount of product molecule produced by the AAV vector particle to an amount of product molecule produced by a viral vector reference standard (e.g. positive control). In certain embodiments, the MOI of the AAV vector particle (and correspdonding potency measurement) is determined using qPCR. In certain embodiments, the amount of product molecule produced by the viral vector reference standard (and correspdonding potency measurement) is based on a MOI determined using ddPCR.

The methods described herein can be performed by utilizing any of a wide range cell assay formats, including, but not limited to cell plates, e.g. ,24-well plates, 48-well plates, 96-well plates, or 384-well plates, individual cell culture plates, or flasks, for example T-flasks or shaker flasks.

In some embodiments, data is analyzed using four parameter logistic regression analysis according to the following equation:

$$Absorbance = D + \frac{A - D}{1 + {( \frac{Concentration}{C} )}^{B} ;}$$

where A is the upper asymptote (“Top”); B is the slope of dynamic range (“Hillslope”); C is the EC₅₀; and D is the lower asymptote (“Bottom”). In some embodiments, a dose response curve is fit to a four-parameter curve analysis. In some embodiments, the relative potency of different samples can be expressed as a value or a shift in the half-maximal effective concentration (EC50) according to four-parameter curve analysis. The linearity of the method allows for accurate comparison of batch-to-batch potency, ensuring consistency.

### III. Compositions and Formulations

### General

Gene therapy drug products (such as rAAV particles) are challenging to incorporate into composition and formulations due to their limited stability in the liquid state and a high propensity for large-scale aggregation at low concentrations. Gene therapy drug products are often delivered directly to treatment areas (including CNS tissue); which requires that excipients and formulation parameters be compatible with tissue function, microenvironment, and volume restrictions.

According to the present disclosure, AAV particles may be prepared as, or included in, pharmaceutical compositions. It will be understood that such compositions necessarily include one or more active ingredients and, most often, one or more pharmaceutically acceptable excipients.

Relative amounts of the active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between .5 and 50%, between 1-30%, between 5-80%, or at least 80% (w/w) active ingredient.

In certain embodiments, the AAV particle pharmaceutical compositions described herein may include at least one payload of the present disclosure. As a non-limiting example, the pharmaceutical compositions may contain an AAV particle with 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In certain embodiments, compositions are administered to humans, human patients or subj ects.

Formulations of the present disclosure can include, without limitation, saline, liposomes, lipid nanoparticles, polymers, peptides, proteins, cells transfected with AAV particles (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to an AAV particle carrying a payload region encoding the polynucleotide or polypeptides of the present disclosure or to the end product encoded by a viral genome of an AAV particle as described herein.

In some embodiments, the formulations may comprise at least one inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of the AAV particles and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In certain embodiments, formulations of the present disclosure are aqueous formulations (i.e. formulations which include water). In certain embodiments, formulations of the present disclosure include water, sanitized water, or Water-for-injection (WFI).

In certain embodiments, the AAV particles of the present disclosure may be formulated in PBS with 0.001%-0.1% (w/v) of Poloxamer 188 (e.g. Pluronic F-68) at a pH of about 7.0.

In certain embodiments, the AAV formulations described herein may contain sufficient AAV particles for expression of at least one expressed functional payload. As a non-limiting example, the AAV particles may contain viral genomes encoding 1, 2, 3, 4 or 5 functional payloads.

According to the present disclosure AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

In certain embodiments, the AAV formulations described herein may include a buffering system which includes phosphate, Tris, and/or Histidine. The buffering agents of phosphate, Tris, and/or Histidine may be independently used in the formulation in a range of 2-12 mM.

Formulations of the present disclosure can be used in any step of producing, processing, preparing, storing, expanding, or administering AAV particles and viral vectors of the present disclosure. In certain embodiments, pharmaceutical formulations and components can be use in AAV production, AAV processing, AAV clarification, AAV purification, and AAV finishing systems of the present disclosure, all of which can be pre-rinsed, packed, equilibrated, flushed, processed, eluted, washed or cleaned with formulations known to those in the art, including AAV pharmaceutical, processing and storage formulations of the present disclosure. Excipients and Diluents

The AAV particles of the present disclosure can be formulated into a pharmaceutical composition which includes one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the present disclosure.

Relative amounts of the active ingredient (e.g. AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. In certain embodiments, the composition may comprise between 0.001% and 99% (w/w) of the active ingredient. By way of example, the composition may comprise between 0.001% and 100%, e.g., between 0.5 and 50%, between 1-30%, between 5-80%, or at least 80% (w/w) active ingredient. In certain embodiments, the composition may comprise between 0.001% and 99% (w/w) of the excipients and diluents. By way of example, the composition may comprise between 0.001% and 100%, e.g., between 0.5 and 50%, between 1-30%, between 5-80%, or at least 80% (w/w) excipients and diluents.

In certain embodiments, a pharmaceutically acceptable excipient may be at least 95%, at least 96%, at least 97%, at least 98%, at least 99%, or 100% pure. In certain embodiments, an excipient is approved for use for humans and for veterinary use. In certain embodiments, an excipient may be approved by United States Food and Drug Administration. In certain embodiments, an excipient may be of pharmaceutical grade. In certain embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary excipients and diluents which can be included in formulations of the present disclosure include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary excipients and diluents which can be included in formulations of the present disclosure include, but are not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, Dl-; Alpha-Tocopherol, Dl-; Aluminum Acetate; Aluminum Chlorhydroxy Allantoinate; Aluminum Hydroxide; Aluminum Hydroxide—Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, Dl-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate—Butyl Methacrylate—Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, Dl-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor FIG. 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O F1-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, Dl-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate—Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa·S); Hypromellose 2910 (15000 Mpa·S); Hypromelloses; Imidurea; Iodine; lodoxamic Acid; lofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate—Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol—Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystalline Wax; Mineral Oil; Mono And Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride): Sebacic Acid; Poly(Dimethylsiloxane/Methylvinylsiloxane/Methylhydrogensiloxane) Dimethylvinyl Or Dimethylhydroxy Or Trimethyl Endblocked; Poly(Dl-Lactic-Co-Glycolic Acid), (50:50; Poly(Dl-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene—Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl; Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, Dl-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical formulations of AAV particles disclosed herein may include cations or anions. In certain embodiments, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations of the present disclosure may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid).

In certain embodiments, additional excipients that may be used in formulating the pharmaceutical composition may include magnesium chloride (MgC12), arginine, sorbitol, and/or trehalose.

Formulations of the present disclosure may include at least one excipient and/or diluent in addition to the AAV particle. The formulation may include 1, 2, 3, 4, 5, 6, 7, 8, 9, 10, or more than 10 excipients and/or diluents in addition to the AAV particle.

In certain embodiments, the formulation may include, but is not limited to, phosphate-buffered saline (PBS). As a non-limiting example, the PBS may include sodium chloride, potassium chloride, disodium phosphate, monopotassium phosphate, and distilled water. In some instances, the PBS does not contain potassium or magnesium. In other instances, the PBS contains calcium and magnesium.

### Formulation Properties

In certain embodiments, the formulation has been optimized to have a specific pH, osmolality, concentration, concentration of AAV particle, and/or total dose of AAV particle.

pH

In certain embodiments, the formulation may be optimized for a specific pH. In certain embodiments, the formulation may include a pH buffering agent (also referred to herein as “buffering agent”) which is a weak acid or base that, when used in the formulation, maintains the pH of the formulation near a chosen value even after another acid or base is added to the formulation. The pH of the formulation may be, but is not limited, to 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, and 14.

In certain embodiments, the formulation may be optimized for a specific pH range. The pH range may be, but is not limited to, 0-4, 1-5, 2-6, 3-7, 4-8, 5-9, 6-10, 7-11, 8-12, 9-13, 10-14, 0-1.5, 1-2.5, 2-3.5, 3-4.5, 4-5.5, 5-6.5, 6-7.5, 7-8.5, 8-9.5, 9-10.5, 10-11.5, 11-12.5, 12-13.5, 0-1, 1-2, 2-3, 3-4, 4-5, 5-6, 6-7, 7-8, 8-9, 9-10, 10-11, 11-12, 12-13, 13-14, 0-0.5, 0.5-1, 1-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.2-8.2, 7.2-7.6, 7.3-7.7, 7.5-8, 7.8-8.2, 8-8.5, 8.5-9, 9-9.5, 9.5-10, 10-10.5, 10.5-11, 11-11.5, 11.5-12, 12-12.5, 12.5-13, 13-13.5, or 13.5-14.

In certain embodiments, the pH of the formulation is between 6 and 8.5.

In certain embodiments, the pH of the formulation is between 7 and 8.5

In certain embodiments, the pH of the formulation is between 7 and 7.6.

In certain embodiments, the pH of the formulation is 7.

In certain embodiments, the pH of the formulation is 7.1.

In certain embodiments, the pH of the formulation is 7.2.

In certain embodiments, the pH of the formulation is 7.3.

In certain embodiments, the pH of the formulation is 7.4.

In certain embodiments, the pH of the formulation is 7.5.

In certain embodiments, the pH of the formulation is 7.6.

In certain embodiments, the pH of the formulation is 7.7.

In certain embodiments, the pH of the formulation is 7.8.

In certain embodiments, the pH of the formulation is 7.9.

In certain embodiments, the pH of the formulation is 8.

In certain embodiments, the pH of the formulation is 8.1.

In certain embodiments, the pH of the formulation is 8.2.

In certain embodiments, the pH of the formulation is 8.3.

In certain embodiments, the pH of the formulation is 8.4.

In certain embodiments, the pH of the formulation is 8.5.

In certain embodiments, the pH is determined when the formulation is at 5° C.

In certain embodiments, the pH is determined when the formulation is at 25° C.

Suitable buffering agents may include, but not limited to, Tris HCl, Tris base, sodium phosphate (monosodium phosphate and/or disodium phosphate), potassium phosphate (monopotassium phosphate and/or dipotassium phosphate), histidine, boric acid, citric acid, glycine, HEPES (4-(2-hydroxyethyl)-1-piperazineethanesulfonic acid), and MOPS (3-(N-morpholino)propanesulfonic acid).

Concentration of buffering agents in the formulation may be between 1-50 mM, between 1-25 mM, between 5-30 mM, between 5-20 mM, between 5-15 mM, between 10-40 mM, or between 15-30 mM. Concentration of buffering agents in the formulation may be about 1 mM, 5 mM, 7.5 mM, 10 mM, 12.5 mM, 15 mM, 20 mM, 25 mM, 30 mM, 35 mM, 40 mM, or 50 mM.

In some embodiments, the formulation may include, but is not limited to, phosphate-buffered saline (PBS). As a non-limiting example, the PBS may include sodium chloride, potassium chloride, disodium phosphate, monopotassium phosphate, and distilled water. In some instances, the PBS does not contain potassium or magnesium. In other instances, the PBS contains calcium and magnesium.

In some embodiments, buffering agents used in the formulations of pharmaceutical compositions described herein may comprise sodium phosphate (monosodium phosphate and/or disodium phosphate). As a non-limiting example, sodium phosphate may be adjusted to a pH (at 5° C.) within the range of 7.4±0.2. In some embodiments, buffering agents used in the formulations of pharmaceutical compositions described herein may comprise Tris base. Tris base may be adjusted with hydrochloric acid to any pH within the range of 7.1 and 9.1. As a non-limiting example, Tris base used in the formulations described herein may be adjusted to 8.0±0.2. As a non-limiting example, Tris base used in the formulations described herein may be adjusted to 7.5±0.2.

### Osmolality

In certain embodiments, the formulation may be optimized for a specific osmolality. The osmolality of the formulation may be, but is not limited to, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mOsm/kg (milliosmoles/kg).

In certain embodiments, the formulation may be optimized for a specific range of osmolality. The range may be, but is not limited to, 350-360, 360-370, 370-380, 380-390, 390-400, 400-410, 410-420, 420-430, 430-440, 440-450, 450-460, 460-470, 470-480, 480-490, 490-500, 350-370, 360-380, 370-390, 380-400, 390-410, 400-420, 410-430, 420-440, 430-450, 440-460, 450-470, 460-480, 470-490, 480-500, 350-375, 375-400, 400-425, 425-450, 450-475, 475-500, 350-380, 360-390, 370-400, 380-410, 390-420, 400-430, 410-440, 420-450, 430-460, 440-470, 450-480, 460-490, 470-500, 350-390, 360-400, 370-410, 380-420, 390-430, 400-440, 410-450, 420-460, 430-470, 440-480, 450-490, 460-500, 350-400, 360-410, 370-420, 380-430, 390-440, 400-450, 410-460, 420-470, 430-480, 440-490, 450-500, 350-410, 360-420, 370-430, 380-440, 390-450, 400-460, 410-470, 420-480, 430-490, 440-500, 350-420, 360-430, 370-440, 380-450, 390-460, 400-470, 410-480, 420-490, 430-500, 350-430, 360-440, 370-450, 380-460, 390-470, 400-480, 410-490, 420-500, 350-440, 360-450, 370-460, 380-470, 390-480, 400-490, 410-500, 350-450, 360-460, 370-470, 380-480, 390-490, 400-500, 350-460, 360-470, 370-480, 380-490, 390-500, 350-470, 360-480, 370-490, 380-500, 350-480, 360-490, 370-500, 350-490, 360-500, or 350-500 mOsm/kg.

In certain embodiments, the osmolality of the formulation is between 350-500 mOsm/kg.

In certain embodiments, the osmolality of the formulation is between 400-500 mOsm/kg

In certain embodiments, the osmolality of the formulation is between 400-480 mOsm/kg.

In certain embodiments, the osmolality is 395 mOsm/kg.

In certain embodiments, the osmolality is 413 mOsm/kg.

In certain embodiments, the osmolality is 420 mOsm/kg.

In certain embodiments, the osmolality is 432 mOsm/kg.

In certain embodiments, the osmolality is 447 mOsm/kg.

In certain embodiments, the osmolality is 450 mOsm/kg.

In certain embodiments, the osmolality is 452 mOsm/kg.

In certain embodiments, the osmolality is 459 mOsm/kg.

In certain embodiments, the osmolality is 472 mOsm/kg.

In certain embodiments, the osmolality is 490 mOsm/kg.

In certain embodiments, the osmolality is 496 mOsm/kg.

### Concentration of AAV Particle

In certain embodiments, the concentration of AAV particle in the formulation may be between about 1×10⁶ VG/ml and about 1×10¹⁶ VG/ml. As used herein, “VG/ml” represents vector genomes (VG) per milliliter (ml). VG/ml also may describe genome copy per milliliter or DNase resistant particle per milliliter.

In certain embodiments, the formulation may include an AAV particle concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1 ×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹²,4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8 ×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/ml.

In certain embodiments, the concentration of AAV particle in the formulation is between 1×10¹¹ and 5×10¹³, between 1×10¹² and 5 ×10¹², between 2 ×10¹² and 1 ×10¹³, between 5×10¹² and 1 ×10¹³, between 1 ×10¹³ and 2 ×10¹³, between 2 ×10¹³ and 3 ×10¹³, between 2 ×10¹³ and 2.5 ×10¹³, between 2.5 ×10¹³ and 3 ×10¹³, or no more than 5×10¹³ VG/ml.

In certain embodiments, the concentration of AAV particle in the formulation is 2.7×10¹¹ VG/ml.

In certain embodiments, the concentration of AAV particle in the formulation is 9×10¹¹ VG/ml.

In certain embodiments, the concentration of AAV particle in the formulation is 1.2×10¹² VG/ml.

In certain embodiments, the concentration of AAV particle in the formulation is 2.7×10¹² VG/ml.

In certain embodiments, the concentration of AAV particle in the formulation is 4×10¹² VG/ml.

In certain embodiments, the concentration of AAV particle in the formulation is 6×10¹² VG/ml.

In certain embodiments, the concentration of AAV particle in the formulation is 7.9×10¹² VG/ml.

In certain embodiments, the concentration of AAV particle in the formulation is 8×10¹² VG/ml.

In certain embodiments, the concentration of AAV particle in the formulation is lx10¹³ VG/ml.

In certain embodiments, the concentration of AAV particle in the formulation is 1.8×10¹³ VG/ml.

In certain embodiments, the concentration of AAV particle in the formulation is 2.2×10¹³ VG/ml.

In certain embodiments, the concentration of AAV particle in the formulation is 2.7×10¹³ VG/ml.

In certain embodiments, the concentration of AAV particle in the formulation is 3.5×10¹³ VG/ml.

In certain embodiments, the concentration of AAV particle in the formulation is 2.7-3.5×10¹³ VG/ml.

In certain embodiments, the concentration of AAV particle in the formulation is 7.0×10¹³ VG/ml.

In certain embodiments, the concentration of AAV particle in the formulation is 5.0×10¹² VG/mL

In certain embodiments, the concentration of AAV particle in the formulation may be between about 1×10⁶ total capsid/mL and about 1×10¹⁶ total capsid/ml. In certain embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/ml.

### Total Dose of AAV Particle

In certain embodiments, the total dose of the AAV particle in the formulation may be between about 1×10⁶ VG and about 1×10¹⁶ VG. In certain embodiments, the formulation may include a total dose of AAV particle of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1 ×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹²,4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8 ×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

In certain embodiments, the total dose of AAV particle in the formulation is between 1×10¹¹ and 5×10¹³VG.

In certain embodiments, the total dose of AAV particle in the formulation is between 1×10¹¹ and 2×10¹⁴ VG.

In certain embodiments, the total dose of AAV particle in the formulation is 1.4×10¹¹ VG.

In certain embodiments, the total dose of AAV particle in the formulation is 4.5×10¹¹ VG.

In certain embodiments, the total dose of AAV particle in the formulation is 6.8×10¹¹ VG.

In certain embodiments, the total dose of AAV particle in the formulation is 1.4×10¹² VG.

In certain embodiments, the total dose of AAV particle in the formulation is 2.2×10¹² VG.

In certain embodiments, the total dose of AAV particle in the formulation is 4.6×10¹¹ VG.

In certain embodiments, the total dose of AAV particle in the formulation is 9.2×10¹² VG.

In certain embodiments, the total dose of AAV particle in the formulation is 1.0×10¹³ VG.

In certain embodiments, the total dose of AAV particle in the formulation is 2.3×10¹³ VG.

### Injectable Formulations

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Depot Formulations

In certain embodiments of the present disclosure, AAV particle formulations of the present disclosure are formulated in depots for extended release. Generally, specific organs or tissues (“target tissues”) are targeted for administration.

In certain embodiments of the disclosure, pharmaceutical compositions, AAV particle formulations of the present disclosure are spatially retained within or proximal to target tissues. Provided are methods of providing pharmaceutical compositions, AAV particle formulations, to target tissues of mammalian subjects by contacting target tissues (which comprise one or more target cells) with pharmaceutical compositions, AAV particle formulations, under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, AAV particle formulations, that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions, AAV particle formulations, administered to subjects are present intracellularly at a period of time following administration.

Certain aspects of the disclosure are directed to methods of providing pharmaceutical compositions, AAV particle formulations of the present disclosure to a target tissues of mammalian subjects, by contacting target tissues (comprising one or more target cells) with pharmaceutical compositions, AAV particle formulations under conditions such that they are substantially retained in such target tissues. Pharmaceutical compositions, AAV particles comprise enough active ingredient such that the effect of interest is produced in at least one target cell.

### IV. Administration and Use

### General

The present disclosure provides a method for treating a disease, disorder and/or condition in a mammalian subject, including a human subject, comprising administering to the subject any of the viral particles or formulations described herein or administering to the subject any of the described compositions, including pharmaceutical compositions or formulations, described herein.

In certain embodiments, administration of the formulated AAV particles to a subject with not change the course of the underlying disease but will ameliorate symptoms in a subject.

In certain embodiments, the viral particles of the present disclosure are administered to a subject prophylactically.

In certain embodiments, the viral particles of the present disclosure are administered to a subject having at least one of the diseases described herein.

In certain embodiments, the viral particles of the present disclosure are administered to a subject to treat a disease or disorder described herein. The subject may have the disease or disorder or may be at-risk to developing the disease or disorder.

The present disclosure provides a method for administering to a subject in need thereof, including a human subject, a therapeutically effective amount of the AAV particles of the present disclosure to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

In certain embodiments, various non-infectious diseases, including neurological diseases, may be treated with pharmaceutical compositions of the present disclosure. AAV particles, especially blood brain barrier crossing AAV particles of the present disclosure, are particularly useful in treating various neurological diseases. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia -Multi-Infarct, Dementia—Semantic, Dementia—Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy—Congenital, Myopathy -Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O′Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain -Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

The present disclosure additionally provides a method for treating neurological disorders in a mammalian subject, including a human subject, comprising administering to the subject any of the AAV particles or pharmaceutical compositions of the present disclosure. In certain embodiments, the AAV particle is a blood brain barrier crossing particle. In certain embodiments, neurological disorders treated according to the methods described herein include, but are not limited to Amyotrophic lateral sclerosis (ALS), Huntington's Disease (HD), Parkinson's Disease (PD), and/or Friedreich's Ataxia (FA).

### Administration

The AAV particles comprising a nucleic acid sequence encoding the siRNA molecules of the present disclosure may be administered by any route which results in a therapeutically effective outcome. These include, but are not limited to, within the parenchyma of an organ such as, but not limited to, a brain (e.g., intraparenchymal), corpus striatum (intrastriatal), enteral (into the intestine), gastroenteral, epidural, oral (by way of the mouth), transdermal, peridural, intracerebral (into the cerebrum), intracerebroventricular (into the cerebral ventricles), subpial (under the pia), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraganglionic (into the ganglion), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In specific embodiments, compositions of AAV particles comprising a nucleic acid sequence encoding the siRNA molecules of the present disclosure may be administered in a way which facilitates the vectors or siRNA molecule to enter the central nervous system and penetrate into medium spiny and/or cortical neurons and/or astrocytes.

In some embodiments, the AAV particles comprising a nucleic acid sequence encoding the siRNA molecules of the present disclosure may be administered by intramuscular injection.

In some embodiments, the AAV particles comprising a nucleic acid sequence encoding the siRNA molecules of the present disclosure may be administered via intraparenchymal injection.

In some embodiments, the AAV particles comprising a nucleic acid sequence encoding the siRNA molecules of the present disclosure may be administered via intraparenchymal injection and intrathecal injection.

In some embodiments, the AAV particles comprising a nucleic acid sequence encoding the siRNA molecules of the present disclosure may be administered via intrastriatal injection.

In some embodiments, the AAV particles comprising a nucleic acid sequence encoding the siRNA molecules of the present disclosure may be administered via intrastriatal injection and another route of administration described herein.

In some embodiments, AAV particles that express siRNA duplexes of the present disclosure may be administered to a subject by peripheral injections (e.g., intravenous) and/or intranasal delivery. It was disclosed in the art that the peripheral administration of AAV particles for siRNA duplexes can be transported to the central nervous system, for example, to the neurons (e.g., U. S. Patent Publication Nos. 20100240739; and 20100130594; the content of each of which is incorporated herein by reference in their entirety).

In other embodiments, compositions comprising at least one AAV particle comprising a nucleic acid sequence encoding the siRNA molecules of the present disclosure may be administered to a subject by intracranial delivery (See, e.g., U.S. Pat. No. 8,119,611; the content of which is incorporated herein by reference in its entirety).

The AAV particle comprising a nucleic acid sequence encoding the siRNA molecules of the present disclosure may be administered in any suitable form, either as a liquid solution or suspension, as a solid form suitable for liquid solution or suspension in a liquid solution. The siRNA duplexes may be formulated with any appropriate and pharmaceutically acceptable excipient.

The AAV particle comprising a nucleic acid sequence encoding the siRNA molecules of the present disclosure may be administered in a “therapeutically effective” amount, i.e., an amount that is sufficient to alleviate and/or prevent at least one symptom associated with the disease, or provide improvement in the condition of the subject.

In some embodiments, the AAV particle may be administered to the cisterna magna in a therapeutically effective amount to transduce medium spiny neurons, cortical neurons and/or astrocytes. As a non-limiting example, the vector may be administered intrathecally.

In some embodiments, the AAV particle may be administered using intrathecal infusion in a therapeutically effective amount to transduce medium spiny neurons, cortical neurons and/or astrocytes. As a non-limiting example, the vector may be administered intrathecally.

In some embodiments, the AAV particle comprising a modulatory polynucleotide may be formulated. As a non-limiting example, the baricity and/or osmolality of the formulation may be optimized to ensure optimal drug distribution in the central nervous system or a region or component of the central nervous system.

In some embodiments, the AAV particle comprising a modulatory polynucleotide may be delivered to a subject via a single route of administration.

In some embodiments, the AAV particle comprising a modulatory polynucleotide may be delivered to a subject via a multi-site route of administration. A subject may be administered the AAV particle comprising a modulatory polynucleotide at 2, 3, 4, 5 or more than 5 sites.

In some embodiments, a subject may be administered the AAV particle comprising a modulatory polynucleotide described herein using a bolus injection.

In some embodiments, a subject may be administered the AAV particle comprising a modulatory polynucleotide described herein using sustained delivery over a period of minutes, hours or days. The infusion rate may be changed depending on the subject, distribution, formulation or another delivery parameter.

In some embodiments, the AAV particle described herein is administered via putamen and caudate infusion. As a non-limiting example, the dual infusion provides a broad striatal distribution as well as a frontal and temporal cortical distribution.

In some embodiments, the AAV particle is AAV-DJ8 which is administered via unilateral putamen infusion. As a non-limiting example, the distribution of the administered AAV-DJ8 is similar to the distribution of AAV1 delivered via unilateral putamen infusion.

In some embodiments, the AAV particle described herein is administered via intrathecal (IT) infusion at C1. The infusion may be for 1, 2, 3, 4, 6, 7, 8, 9, 10, 11, 12, 13, 14, 15 or more than 15 hours.

In some embodiments, the selection of subjects for administration of the AAV particle described herein and/or the effectiveness of the dose, route of administration and/or volume of administration may be evaluated using imaging of the perivascular spaces (PVS) which are also known as Virchow-Robin spaces. PVS surround the arterioles and venules as they perforate brain parenchyma and are filled with cerebrospinal fluid (CSF)/interstitial fluid. PVS are common in the midbrain, basal ganglia, and centrum semiovale. While not wishing to be bound by theory, PVS may play a role in the normal clearance of metabolites and have been associated with worse cognition and several disease states including Parkinson's disease. PVS are usually are normal in size but they can increase in size in a number of disease states. Potter et al. (Cerebrovasc Dis. 2015 January; 39(4): 224-231; the contents of which are herein incorporated by reference in its entirety) developed a grading method where they studied a full range of PVS and rated basal ganglia, centrum semiovale and midbrain PVS. They used the frequency and range of PVS used by Mac and Lullich et al. (J Neurol Neurosurg Psychiatry. 2004 Nov;75(11):1519-23; the contents of which are herein incorporated by reference in its entirety) and Potter et al. gave 5 ratings to basal ganglia and centrum semiovale PVS: 0 (none), 1 (1-10), 2 (11-20), 3 (21-40) and 4 (>40) and 2 ratings to midbrain PVS: 0 (non-visible) or 1 (visible). The user guide for the rating system by Potter et al. can be found at: www.sbirc.ed.ac.uk/documents/epvs-rating-scale-user-guide.pdf.

In some embodiments, AAV particles described herein is administered via thalamus infusion. Infusion into the thalamus may be bilateral or unilateral.

In some embodiments, AAV particles described herein are administered via putamen infusion. Infusion into the thalamus may be bilateral or unilateral.

In some embodiments, AAV particles described herein are administered via putamen and thalamus infusion. Dual infusion into the putamen and thalamus may maximize brain distribution via axonal transport to cortical areas. Evers et al. observed positive transduction of neurons in the motor cortex and part of the parietal cortex after bilateral injections of AAV5-GFP into the putamen and thalamus of tgHD minipigs (Molecular Therapy (2018), doi: 10.1016/j.ymthe.2018.06.021). Infusion into the putamen and thalamus may be independently bilateral or unilateral. As a non-limiting example, AAV particles may be infused into the putamen and thalamus from both sides of the brain. As another non-limiting example, AAV particles may be infused into the left putamen and left thalamus, or right putamen and right thalamus. As yet another non-limiting example, AAV particles may be infused into the left putamen and right thalamus, or right putamen and left thalamus. Dual infusion may occur consecutively or simultaneously.

In some embodiments, the AAV particle comprising a modulatory polynucleotide may be delivered to a subject in the absence of gene therapy-related changes in body weight.

In some embodiments, the AAV particle comprising a modulatory polynucleotide may be delivered to a subject in the absence of gene therapy-related clinical signs, including but not limited to incoordination, inappetence, decreased feeding, and overall weakness.

In some embodiments, the AAV particle comprising a modulatory polynucleotide may be delivered to a subject in the absence of gene therapy-related changes to blood of a subject. In certain embodiments, the changes in blood of a subject are serum chemistry, and coagulation parameters.

In some embodiments, the AAV particle comprising a modulatory polynucleotide may be delivered to a subject in the absence of pathological changes to a tissue of a subject (e.g., brain of the subject). In certain embodiments the pathological change is a gross pathological change, such as, but not limited to, atrophy. In certain embodiments, the pathological change is a histopathological change, including but not limited to, target specific (e.g., HTT) inclusions. Use of AAV particles encoding protein payloads

Provided in the present disclosure are methods for introducing into cells the AAV particles manufactured according to the methods and systems of the present disclosure, the methods comprising introducing into said cells any of the vectors in an amount sufficient for an increase in the production of target mRNA and protein to occur. In some aspects, the cells may be muscle cells, stem cells, neurons such as but not limited to, motor, hippocampal, entorhinal, thalamic or cortical neurons, and glial cells such as astrocytes or microglia.

Disclosed in the present disclosure are methods for treating neurological disease associated with insufficient function/presence of a target protein in a subject in need of treatment. The method optionally includes administering to the subject a therapeutically effective amount of a composition comprising AAV particles of the present disclosure. As a non-limiting example, the AAV particles can increase target gene expression, increase target protein production, and thus reduce one or more symptoms of neurological disease in the subject such that the subject is therapeutically treated.

In certain embodiments, the AAV particle of the present disclosure comprising a nucleic acid encoding a protein payload includes an AAV capsid that allows for transmission across the blood brain barrier after intravenous administration.

In certain embodiments, the composition comprising the AAV particles of the present disclosure is administered to the central nervous system of the subject via systemic administration. In certain embodiments, the systemic administration is intravenous injection.

In certain embodiments, the composition comprising the AAV particles of the present disclosure is administered to the central nervous system of the subject. In certain embodiments, the composition comprising the AAV particles of the present disclosure is administered to a tissue of a subject (e.g., brain of the subject).

In certain embodiments, the composition comprising the AAV particles of the present disclosure is administered to the central nervous system of the subject via intraparenchymal injection. Non-limiting examples of intraparenchymal injections include intrathalamic, intrastriatal, intrahippocampal or targeting the entorhinal cortex.

In certain embodiments, the composition comprising the AAV particles of the present disclosure is administered to the central nervous system of the subject via intraparenchymal injection and intrathecal injection.

In certain embodiments, the AAV particles of the present disclosure may be delivered into specific types of targeted cells, including, but not limited to, hippocampal, cortical, motor or entorhinal neurons; glial cells including oligodendrocytes, astrocytes and microglia; and/or other cells surrounding neurons such as T cells.

In certain embodiments, the AAV particles of the present disclosure may be delivered to neurons in the striatum (e.g. putamen) and/or cortex.

In certain embodiments, the AAV particles of the present disclosure may be used as a therapy for neurological disease.

In certain embodiments, the AAV particles of the present disclosure may be used to increase target protein and reduce symptoms of neurological disease in a subject. The increase of target protein and/or the reduction of symptoms of neurological disease may be, independently, altered (increased for the production of target protein and reduced for the symptoms of neurological disease) by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

### Use of AAV Particles Comprising RNAi Polynucleotides

Provided in the present disclosure are methods for introducing the AAV particles, comprising a nucleic acid sequence encoding the siRNA molecules of the present disclosure into cells, the method comprising introducing into said cells any of the vectors in an amount sufficient for degradation of a target mRNA to occur, thereby activating target-specific RNAi in the cells. In some aspects, the cells may be muscle cells, stem cells, neurons such as but not limited to, motor, hippocampal, entorhinal, thalamic or cortical neurons, and glial cells such as astrocytes or microglia.

Disclosed in the present disclosure are methods for treating neurological diseases associated with dysfunction of a target protein in a subject in need of treatment. The method optionally includes administering to the subject a therapeutically effective amount of a composition comprising AAV particles comprising a nucleic acid sequence encoding the siRNA molecules of the present disclosure. As a non-limiting example, the siRNA molecules can silence target gene expression, inhibit target protein production, and reduce one or more symptoms of neurological disease in the subject such that the subject is therapeutically treated.

In certain embodiments, the composition comprising the AAV particles of the present disclosure comprising a nucleic acid sequence encoding siRNA molecules include an AAV capsid that allows for transmission across the blood brain barrier after intravenous administration.

In certain embodiments, the composition comprising the AAV particles comprising a nucleic acid sequence encoding the siRNA molecules of the present disclosure is administered to the central nervous system of the subject. In certain embodiments, the composition comprising the AAV particles comprising a nucleic acid sequence encoding the siRNA molecules of the present disclosure is administered to a tissue of a subject (e.g., brain of the subject).

In certain embodiments, the composition comprising the AAV particles comprising a nucleic acid sequence encoding the siRNA molecules of the present disclosure is administered to the central nervous system of the subject via systemic administration. In certain embodiments, the systemic administration is intravenous injection.

In certain embodiments, the composition comprising the AAV particles comprising a nucleic acid sequence encoding the siRNA molecules of the present disclosure is administered to the central nervous system of the subject via intraparenchymal injection. Non-limiting examples of intraparenchymal injections include intrathalamic, intrastriatal, intrahippocampal or targeting the entorhinal cortex.

In certain embodiments, the composition comprising the AAV particles comprising a nucleic acid sequence encoding the siRNA molecules of the present disclosure is administered to the central nervous system of the subject via intraparenchymal injection and intrathecal injection.

In certain embodiments, the AAV particles comprising a nucleic acid sequence encoding the siRNA molecules of the present disclosure may be delivered into specific types of targeted cells, including, but not limited to, hippocampal, cortical, motor or entorhinal neurons; glial cells including oligodendrocytes, astrocytes and microglia; and/or other cells surrounding neurons such as T cells.

In certain embodiments, the AAV particles comprising a nucleic acid sequence encoding the siRNA molecules of the present disclosure may be delivered to neurons in the striatum and/or cortex.

In certain embodiments, the AAV particles comprising a nucleic acid sequence encoding the siRNA molecules of the present disclosure may be used as a therapy for neurological disease.

In certain embodiments, the AAV particles comprising a nucleic acid sequence encoding the siRNA molecules of the present disclosure may be used as a therapy for Amyotrophic Lateral Sclerosis.

In certain embodiments, the AAV particles comprising a nucleic acid sequence encoding the siRNA molecules of the present disclosure may be used as a therapy for Huntington's Disease.

In certain embodiments, the AAV particles comprising a nucleic acid sequence encoding the siRNA molecules of the present disclosure may be used as a therapy for Parkinson's Disease.

In certain embodiments, the AAV particles comprising a nucleic acid sequence encoding the siRNA molecules of the present disclosure may be used as a therapy for Friedreich's Ataxia.

In certain embodiments, the AAV particles comprising a nucleic acid sequence encoding the siRNA molecules of the present disclosure may be used to suppress a target in order to treat neurological disease. Target protein in astrocytes may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. Target protein in astrocytes may be reduced may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, administration of the AAV particles encoding a siRNA of the present disclosure, to a subject may lower target protein levels in a subject. The target protein levels may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may lower the protein levels of a target protein by at least 50%. As a non-limiting example, the AAV particles may lower the proteins levels of a target protein by at least 40%.

### V. Definitions

At various places in the present disclosure, substituents or properties of compounds of the present disclosure are disclosed in groups or in ranges. It is specifically intended that the present disclosure include each and every individual or subcombination of the members of such groups and ranges.

Unless stated otherwise, the following terms and phrases have the meanings described below. The definitions are not meant to be limiting in nature and serve to provide a clearer understanding of certain aspects of the present disclosure.

About: As used herein, the term “about” means +/−10% of the recited value.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” is a virus which includes a capsid and a viral genome with at least one payload region and at least one ITR region. AAV particles of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the present disclosure may have activity and this activity may involve one or more biological events.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the patient. In certain embodiments, they are administered within about 60, 30, 15, 10, 5, or 1 minute of one another. In certain embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals include, but are not limited to, mammals, birds, reptiles, amphibians, fish, and worms. In certain embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, i.e., a polynucleotide vector of baculoviral origin. Systems using BEVs are known as baculoviral expression vector systems (BEVSs).

mBEV or modified BEV: As used herein, a modified BEV is an expression vector of baculoviral origin which has been altered from a starting BEV (whether wild type or artificial) by the addition and/or deletion and/or duplication and/or inversion of one or more: genes; gene fragments; cleavage sites; restriction sites; sequence regions; sequence(s) encoding a payload or gene of interest; or combinations of the foregoing.

Bifunctional: As used herein, the term “bifunctional” refers to any substance, molecule or moiety which is capable of or maintains at least two functions. The functions may affect the same outcome or a different outcome. The structure that produces the function may be the same or different.

BIIC: As used herein a BIIC is a baculoviral infected insect cell.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the protein shell of a virus particle.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which includes an AAV capsid protein can be codon optimized for expression in insect cells or in a particular insect cell such Spodoptera frupperda cells. Codon optimization can be completed using methods and databases known to those in the art.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the present disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle and a delivery agent or excipient.

Fragment: A “fragment,” as used herein, refers to a portion. For example, fragments of proteins may include polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In certain embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or AAV particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the present disclosure. Molecules may be modified in many ways including chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Neurodegeneration: As used herein, the term “neurodegeneration” refers to a pathologic state which results in neural cell death. A large number of neurological disorders share neurodegeneration as a common pathological state. For example, Alzheimer's disease, Parkinson's disease, Huntington's disease, and amyotrophic lateral sclerosis (ALS) all cause chronic neurodegeneration, which is characterized by a slow, progressive neural cell death over a period of several years, whereas acute neurodegeneration is characterized by a sudden onset of neural cell death as a result of ischemia, such as stroke, or trauma, such as traumatic brain injury, or as a result of axonal transection by demyelination or trauma caused, for example, by spinal cord injury or multiple sclerosis. In some neurological disorders, mainly one type of neuronal cell is degenerative, for example, medium spiny neuron degeneration in early HD.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid: As used herein, the term “nucleic acid”, “polynucleotide” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene, or cellular transcript.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

Payload construct: As used herein, “payload construct” is one or more vector construct which includes a polynucleotide region encoding or comprising a payload that is flanked on one or both sides by an inverted terminal repeat (ITR) sequence. The payload construct presents a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile can be used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the present disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In certain embodiments, when referring to a protein or protein module, a region may include a linear sequence of amino acids along the protein or protein module or may include a three-dimensional area, an epitope and/or a cluster of epitopes. In certain embodiments, regions include terminal regions. As used herein, the term “terminal region” refers to regions located at the ends or termini of a given agent. When referring to proteins, terminal regions may include N- and/or C-termini. N-termini refer to the end of a protein comprising an amino acid with a free amino group. C-termini refer to the end of a protein comprising an amino acid with a free carboxyl group. N- and/or C-terminal regions may there for include the N- and/or C-termini as well as surrounding amino acids. In certain embodiments, N- and/or C-terminal regions include from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may include any length of amino acids that includes the N-terminus but does not include the C-terminus. In certain embodiments, C-terminal regions may include any length of amino acids, which include the C-terminus, but do not include the N-terminus.

In certain embodiments, when referring to a polynucleotide, a region may include a linear sequence of nucleic acids along the polynucleotide or may include a three-dimensional area, secondary structure, or tertiary structure. In certain embodiments, regions include terminal regions. As used herein, the term “terminal region” refers to regions located at the ends or termini of a given agent. When referring to polynucleotides, terminal regions may include 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for include the 5′ and 3′ termini as well as surrounding nucleic acids. In certain embodiments, 5′ and 3′ terminal regions include from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may include any length of nucleic acids that includes the 5′ terminus but does not include the 3′ terminus. In certain embodiments, 3′ regions may include any length of nucleic acids, which include the 3′ terminus, but does not include the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle included of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule includes between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In certain embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and in certain embodiments, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, such as a mammal, a human, or a human patient.

Terminal region: As used herein, the term “terminal region” refers to a region on the 5′ or 3′ end of a region of linked nucleosides or amino acids (polynucleotide or polypeptide, respectively).

Terminally optimized: The term “terminally optimized” when referring to nucleic acids means the terminal regions of the nucleic acid are improved in some way, e.g., codon optimized, over the native or wild type terminal regions.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to include a therapeutically effective amount of a particular agent or entity if it includes an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24-hour period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may include adeno-associated virus (AAV) parent or reference sequence. Such parent or reference AAV sequences may serve as an original, second, third or subsequent sequence for engineering vectors. In non-limiting examples, such parent or reference AAV sequences may include any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence . These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” or “viral vector” refers to the nucleic acid sequence(s) encapsulated in an AAV particle. Viral genomes comprise at least one payload region encoding polypeptides or fragments thereof.

### VI. Equivalents and Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### EXAMPLES

### Example 1

### Production of AAV Vectors with a Baculovirus

AAV vector particles (AAV2 capsid with protein payload) was produced in a Sf9/baculovirus system according to the present disclosure. A cell bank was thawed to initiate Sf9 cell culture expansion in EFS AFTM Insect Cell Culture Medium (Expression Systems, LLC). The number of viable Sf9 cells was expanded using a shake flask and WAVE Bioreactor (GE Life Sciences) to enable rolling inoculation into the 200 L single use bioreactor. In the single use bioreactor, Sf9 cells were further expanded at 26-27° C. The AAV vector particles were then produced by infecting the Sf9 cells with baculoviruses (BIIC) which included BIIC-rep2/cap2 and BIIC-payload at 26° C. Chemical lysis of the Sf9 cells was performed at 18-25° C. to release the AAV vector particles from the cell nucleus. The material was clarified by removal of cell debris using immunoaffinity chromatography and anion exchange chromatography. The AAV vector particles were formulated in phosphate buffered saline (PBS) at a target concentration using ultrafiltration (UF) and diafiltration (DF), and the resulting formulation was cleared by nanofiltration and 0.2 μM filtration immediately pior to the final fill. 0.001% pluronic acid (F-68) was be added for a final fill resulting in the drug product.

### Example 2

### Fill and Finish

A Drug Substance was transferred to a Biosafety Cabinet (BSC) and filtered through a 0.22 μm filter (dual-in-line sterilizing grade filters). The filtered Drug Substance pool was then aseptically filled into 2 ml Cryovials utilizing a programmable Peristaltic dispensing pump within the BSC. Product vials were stoppered, seal capped, 100% visually inspected and labeled (at 25° C.), and then stored at ≤−65° C.

### Example 3

### Sample Digestions for ddPCR and qPCR

Samples of cells containing intact rAAV particles were prepared for quantitative polymerase chain reaction (qPCR) and digital droplet polymerase chain reaction (ddPCR). In general, samples were treated as follows: (i) Treatment with DNase to digest any non-encapsidated DNA; (ii) DNase reactions were stopped using EDTA; (iii) AAV capsids were digested with proteinase K (proK); and (iv) Samples were heated to 95° C. to denature proK and to denature any capsids that did not fully digest from the proK treatment.

At least one vector reference standard, if available, was included as an additional sample. Vector reference standard was an rAAV sample having the same DNA region (amplicon) that was to be used for amplification in the qPCR and/or ddPCR method. The equation includes an additional 4 wells to account for dead volume during pipetting.

### PCR Plate Preperation

The total number of wells required for the procedure was calculated using the equation:

Total # of wells=(# of samples×3 replicates+4

95 μL of DNasereaction mixture was required for each well. The DNase reaction mixture was prepared using the following proportions: 3 μL/well of 10 mg/mL DNase I and 92 μL/well qPCR Dnase buffer. 95 μL of DNase reaction mixture is added to each well with a 5 mL repeater pipette. The plate was sealed with clear plate sealing film (Axygen Cat #PCR-TS or equivalent) and placed in a thermocycler. The thermocycler was run on a cycle set to heat to 37° C. for 1 hour followed by a 4° C. hold for up to 24 hours.

### Proteinase K Treatment

The required amount of ProK/EDTA reaction mixture was prepared using the following proportions (125 μL of reaction mixture for each well): 12 μL/well of 10 mg/mL ProteinaseK, 108 μL/well of qPCR ProteinaseK buffer, and 5 μL/well 0.5M EDTA pH 8.

The sealing film was removed from the PCR plate, and 125 μL, of ProK/EDTA reaction mixture was added to each sample well from the DNase treatment with a 5 mL repeater pipette. The plate was sealed with a new clear plate sealing film (Axygen Cat #PCR-TS or equivalent) and placed in a thermocycler. The thermocycler was run a cycle set to heat to 55° C. for 60 minutes, followed by 95° C. for 10 minutes, and then hold at 4° C. for up to 24 hours. Digested samples were analyzed for titer determination by ddPCR and/or qPCR.

### Example 4

### Sample Analysis Using qPCR Assay

Samples of cells containing intact rAAV particles were prepared for quantitative polymerase chain reaction (qPCR) according to Example 3 (“Reaction Plate”). The resulting digested samples in the PCR plate were analyzed using qPCR.

### Sample Dilution

195 μL of 10 mM Tris pH 7.5 was added to wells in a 96-well PCR dilution plate (same number of wells as the Reaction Plate from Example 3). The sealing film was removed from the Reaction Plate and 5 μL of ProK-digested sample was added to the buffer wells in the dilution plate using a 12-well multichannel 10 μL pipette. The dilution wells were mixed (via pipette) no less than 10 times with a larger volume (>100 μL) setting on a pipette.

16 wells were reserved for standard curve dilutions (typically a linearized plasmid or purified DNA sample containing the same amplification sequence as the samples). 180 μL of 10 mM Tris pH 7.5 was added each of the 16 standard wells, and 20 μL of qPCR reference standard was then added to the first two wells in the standard curve section (which was then pipette-mixed with a larger volume (>100 μL) no less than 10 times). 20 μL of the standard from the first two wells was transfered to the next two wells (pipetted from the top of the liquid level in the well) and pipette-mixed no less than 10 times with a larger volume (>100 μL). This serial dilution was repeated five additional times to generate a 7-point standard curve with two no template controls (NTCs).

The reaction plate was then resealed with a new 96-well plate sealing film.

qPCR Master Mix and Plate Loading

qPCR Master Mix was prepared in a biosafety cabinet using the following mixtures:10 μL of Taqman® Fast Advanced Master Mix (2×); 1 μL of 20× Forward Primer/Reverse Primer with Probe; 5 μL Nuclease-free Water. 16 μL/well of qPCR master mix was loaded into a LightCycler® 480 Multiwell Plate 96 plate with a repeat pipette (matching the well pattern to the dilution wells, including the standard curve wells). 4 μL of the sample dilutions and standard curve dilutions were added to the corresponding qPCR plate wells and then pipette-mixed with the same tips used to transfer the samples (different tips for each sample). The plate was then sealed with LightCycler® 480 Sealing Foil. The sealed plate was centrifuged for approximately 30 seconds in a 96-well plate centrifuge and visually inspected to ensure that the liquid in each well was at the bottom of the well.

qPCR Analysis

qPCR analysis was completed using a LightCycler® 480 and corresponding LightCycler® 480 SW software. Samples and standard wells were identified in the software and the samples were analyzed accordingly with a target output of “Absolute quantification/2nd Derivative” data.

qPCR results were analyzed using the “Absolute quantification/2nd Derivative” option with the appropriate standard values from the standard serial dilutions (Wells with values outside of the standard curve range were retested or excluded from titer calculation). Titers were then calculated for each sample using the following calculations:

$$Titer ( \frac{vg}{mL} ) = \frac{qPCRssCopies}{4 μ L} \times \frac{225 μ L}{5 μ L} \times \frac{200 μ L}{5 μ L} \times \frac{1000 μ L}{mL}$$

Average titer values were calculated from sample replicates (along with corresponding standard deviations).

### Example 5

### Sample Analysis Using ddPCR Assay

Samples of cells containing intact rAAV particles were prepared for digital droplet polymerase chain reaction (ddPCR) according to Example 3 (“Reaction Plate”). The resulting digested samples in the PCR plate were analyzed using ddPCR.

### Sample Dilution

A total of four 10-fold dilutions were performed on the proK-digested samples in a 96-well PCR plate V-shaped, cat #60180-P100 (ThermoFisher). The first dilution was performed using 180 μL of Tris buffer and 20 μL of proK digested material for the first dilution. Each of the wells was mixed by pipetting with no less than 10 times a larger volume (i.e., 100 μL). The next three serial dilutions were performed by transferring 20 μL of diluted material into 180 μL of Tris buffer in sebsequent wells. Each of the wells was mixed by pipetting with no less than 10 times a larger volume (i.e., 100 μL) setting on the pipette. Once all of the samples were diluted, the plate(s) containing the proK digested samples were resealed with a new plate sealing film and retained at 4° C. for no more than 72 hours. ddPCR Supermix and loading of PCR plate

Preparation of the ddPCR Supermix (Bio-Rad) and loading of the ddPCR Plate was performed in a BSC or a PCR hood. The ddPCR Supermix (Bio-Rad) was prepared using 12.5 μL of Supermix (probes no dUTP) (Bio-Rad); 1.25 μL of 20× forward primer/reverse primer with probe; and 8.75 μL of nuclease-free water for each well. Alternatively, Taqman ddPCR may be performed, in which a probe is included in the reaction mixture. 22.5 μL of ddPCR Supermix was loaded into an approparite number of wells in an Eppendorf Twin-Tec 96-well Semi-Skirted PCR plate with a Xstream Repeat Pipettor (Eppendorf). At least two negative control wells that only contain the ddPCR Supermix (Bio-Rad) and the buffer were included. 25 μL of buffer control Supermix (Bio-Rad) was loaded with the repeat pipettor into any remaninig wells (to provide multiple of 8).

2.5 μL of the sample dilutions from the Reaction Plate were added to the corresponding ddPCR plate wells and mixed with a multichannel pipette (Rainin multi-channel micropipettes). The plate was sealed with clear plate sealing film (Axygen Cat #PCR-TS or equivalent). The plate was centifuged for approximately 30 seconds in a 96-well plate centrifuge and visually inspected to ensure that the liquid in each well is at the bottom of the well.

Droplet Generation and ddPCR

An Automated Droplet Generation (AutoDG) instrument was used to generate droplets of the samples from each well.

PCR was performed on the AutoDG-processed plate in a thermocycler with the following settings: 95° C. for 10 minutes; cycle 40 times at 94° C. for 30 seconds 60° C. for 1 minute, 60° C. for 5 minutes, and 98° C. for 5 minutes; and a 10° C. hold for no longer than 96 hours.

QuantaSoft software was used to analyze the samples in a QX200™ AutoDG™ Droplet Digital™ PCR System with an automated droplet generator an DX-200, droplet reader (BioRad). To analyze samples using the PCR system, the acceptable concentration range for each well (prior to normalization to the dilution) was 50-4000 copies/μL. Wells outside of this range were excluded when calculating averages and percent (%) CV. If both dilutions (all wells) for a sample are out of this range, the ddPCR measurement was rerun. If the measurement was too high, an additional two 10-fold dilutions were performed and the ddPCR measurement was repeated using these dilutions.

The titers for each sample were calculated using the following equation:

$$Titer ( \frac{vg}{mL} ) = DDPCR Concentration \times \frac{25 μ L}{2.5 μ L} \times \frac{225 μ L}{5 μ L} \times {10}^{dilution} \times \frac{1000 μ L}{mL}$$

For each well, the Poisson Confidence was calculated using the following equation:

$$Poisson Confidence = 100 \times \frac{Poisson Conf Max - Poisson Conf Min}{DDPCR Concentration}$$

Titers from the sample replicates were averaged, and the standard deviation was calculated. Samples that have Poisson Confidence values greater than 15% were not included in averages or standard deviations.

The percent of change of the coefficient of variation (% CV) of the averages was also calculated:

$$\% CV = 100 \% \times \frac{Standard Deviation of Averaged Values}{Average of the Values}$$

The % CV values were observed to be less than or equal to 25%. The average number of positive droplets in the negative control wells was calculated, and the vector reference standard passed the standard titer acceptance criteria. An average no template control ddPCR well was observed to have no more than 100 positive events.

### Example 6

### Determination of Viral Vector Titer

Comparability and variability of the qPCR and ddPCR methods in determining viral vector titer were assessed.

qPCR Assays

Real-time qPCR amplification assays were carried out according to Example 4. Samples were assayed 8 times by three independent operators, with each assay containing two sets of independently generated standard curves to get 16 values for each material. 13 out of 16 standard curves had values that passed acceptance criteria.

A second derivative max method was used to process the resulting data, wherein the fluorescence emission during the qPCR reaction (proportional to the synthesized DNA) was used to visualize and generate amplification plots, with independently defined crossing point-PCR-cycle (Cₚ) values given for each curve. Standard amplification curves were plotted and coupled with the associated Cₚ value and compared to the amplification curves of the samples to determine concentrations. Results of initial standard and reference qPCR assays are shown in Table 1 below. Cp values are given in fluorescence units

TABLE 1
qPCR Reference and Standard results

| qPCR assay | qPCR run 1 | qPCR run 2 |
|---|---|---|
| Average Cₚ of highest conc. Standard | 11.6 | 10.88 |
| Average Cₚ of reference | 18.81 | 18.81 |
| Reference | Reference 1 | Reference 1 |
| Average Reference Titer | 5.75 × 10¹¹ | 3.63 × 10¹¹ |

Four batches of viral vector (Batch 1, 2, 3, 4) were assayed by qPCR and assessed for operator variability (3 independent operators) and batch comparability. Data are shown in Table 2 below as viral genomes/mL (vg/mL). CV indicates coefficient of variation.

TABLE 2
Viral vector titer (qPCR)

| | Batch 1 | Batch 2 | Batch 3 | Batch 4 |
|---|---|---|---|---|
| | (vg/mL) | (vg/mL) | (vg/mL) | (vg/mL) |
| Operator 1 | 4.0 × 10¹² | 4.3 × 10¹² | 3.6 × 10¹² | 4.1 × 10¹² |
| | 2.5 × 10¹² | 2.7 × 10¹² | 2.3 × 10¹² | 2.6 × 10¹² |
| | 1.9 × 10¹² | 2.2 × 10¹² | 1.9 × 10¹² | 2.2 × 10¹² |
| | 2.3 × 10¹² | 2.6 × 10¹² | 2.3 × 10¹² | 2.6 × 10¹² |
| | 2.6 × 10¹² | 2.8 × 10¹² | 2.5 × 10¹² | 2.8 × 10¹² |
| | 2.1 × 10¹² | 2.3 × 10¹² | 2.0 × 10¹² | 2.3 × 10¹² |
| Operator 2 | 3.0 × 10¹² | 3.3 × 10¹² | 2.8 × 10¹² | 3.3 × 10¹² |
| | 2.4 × 10¹² | 2.6 × 10¹² | 2.2 × 10¹³ | 2.6 × 10¹² |
| | 1.8 × 10¹² | 2.0 × 10¹² | 1.7 × 10¹² | 2.0 × 10¹² |
| Operator 3 | 2.3 × 10¹² | 2.4 × 10¹² | 2.1 × 10¹² | 2.4 × 10¹² |
| | 3.0 × 10¹² | 3.1 × 10¹² | 2.8 × 10¹² | 3.1 × 10¹² |
| | 2.1 × 10¹² | 2.3 × 10¹² | 2.0 × 10¹² | 2.3 × 10¹² |
| | 2.8 × 10¹² | 3.1 × 10¹² | 2.7 × 10¹² | 3.1 × 10¹² |
| Average Titer | 2.5 × 10¹² | 2.7 × 10¹² | 2.4 × 10¹² | 2.7 × 10¹² |
| Reference Titer | 3.7 × 10¹² | 3.5 × 10¹² | 3.7 × 10¹² | 3.8 × 10¹² |
| Inter-assay % CV | 23.9 | 22.2 | 21.6 | 21.2 |
| Intra-assay % CV | 3.6 | 2.2 | 2.2 | 2.6 |

Each vector batch demonstrated a range of viral titers (1.7×10¹² to 4.3×10¹²) as determined by qPCR and three independent observers. Titers quantified across batches were substantially similar and no clear operator bias was identified. Inter-assay coefficient of variation (% CV) were higher (21-24%) than intra-assay % CV values (2-4%).

Across the thirteen qualifying qPCR assays performed, similar titer quantifications and patterns were observed (titer range of approximately 1.5×10¹² to 4.5×10¹²). This finding suggests that the potential root cause of qPCR inter-assay variability may derive from the use of standard curve(s).

ddPCR Assays

The same four viral vector batches (Batches 1-4) described above were also assayed using ddPCR according to Example 5. Samples were assayed 8 times by three independent operators, with 6 out of 8 of the assays passing acceptance criteria.

End point ddPCR assays were conducted using water-oil emulsion droplets for determination of absolute copy number as quantified by the ratio of positive to negative droplets in the sample. This ratio was used to generate a raw concentration, without the necessity of a standard curve for quantification. Viral vector titers (vg/ml) are shown in Table 3 below. CV indicates coefficient of variation.

TABLE 3
Viral vector titer (ddPCR)

| | Batch 1 | Batch 2 | Batch 3 | Batch 4 |
|---|---|---|---|---|
| Operator 1 | 2.3 × 10¹² | 2.6 × 10¹² | 2.6 × 10¹² | 2.9 × 10¹² |
| | 2.4 × 10¹² | 2.8 × 10¹² | 2.4 × 10¹² | 2.7 × 10¹² |
| | 2.7 × 10¹² | 3.1 × 10¹² | 2.6 × 10¹² | 3.2 × 10¹² |
| Operator 2 | 2.5 × 10¹² | 2.6 × 10¹² | 2.3 × 10¹² | 2.3 × 10¹² |
| Operator 3 | 2.8 × 10¹² | 3.2 × 10¹² | 2.7 × 10¹³ | 3.0 × 10¹² |
| | 2.3 × 10¹² | 2.8 × 10¹² | 2.5 × 10¹² | 2.9 × 10¹² |
| Average Titer | 2.5 × 10¹² | 2.8 × 10¹² | 2.5 × 10¹² | 2.8 × 10¹² |
| Reference Titer | 3.7 × 10¹² | 3.5 × 10¹² | 3.7 × 10¹² | 3.8 × 10¹² |
| Inter-assay % CV | 7.2 | 7.7 | 6.2 | 9.9 |
| Intra-assay % CV | 7.1 | 5.6 | 3.6 | 4.6 |

Quantification of viral vector titer using ddPCR produced a smaller distribution of viral vector titers (2.3×10¹²to 3.2×10¹²), as compared to quantification by qPCR. As with analysis by qPCR assay, the ddPCR assay analysis did not show any operator bias. Quantification based on ddPCR assay showed inter-assay % CV values (6-10%) of the same magnitude as the intra-assay % CV values (4-7%).

Average titer values for qPCR and ddPCR were comparable for all batches and across all assay runs. ddPCR measurements showed smaller inter-assay % CV values than qPCR measurements. Since double the inter-assay % CV values were observed with qPCR titer vs ddPCR titer, more replicate measurements were used to achieve the same confidence in the absolute titer. Vector genome titer by ddPCR yielded more accurate results but with slightly larger variability. Vector genome titer by qPCR generated more precise results (intra-assay variability), but due to the standard curve, had greater inter-assay variability. qPCR values were used to normalize titers and determine a starting MOI for subsequent potency studies.

Despite showing low inter-assay variability, the inter-assay % CV for qPCR titer assay were double of the one for the ddPCR titer assay, requiring more replicate measurements to achieve comparable confidence in the absolute titers.

Deviation from Mean for qPCR and ddPCR Titer

Deviations from mean were collected for qPCR and ddPCR, as shown in Table 4. qPCR deviations from mean were systematic but appeared to be random for ddPCR.

TABLE 4
Deviation from mean for qPCR and ddPCR titer

| | Batch 1 | Batch 2 | Batch 3 | Batch 4 | Reference |
|---|---|---|---|---|---|
| qPCR | | | | | |
| Run 1 | 0.92 | 0.89 | 0.91 | 0.89 | 0.89 |
| Run 2 | 1.21 | 1.17 | 1.20 | 1.17 | 1.18 |
| Run 3 | 0.86 | 0.85 | 0.87 | 0.86 | 0.82 |
| Run 4 | 1.15 | 1.14 | 1.16 | 1.15 | 1.10 |
| Run 5 | 1.62 | 1.60 | 1.56 | 1.55 | 1.54 |
| Run 6 | 1.02 | 1.01 | 0.99 | 0.98 | 0.97 |
| Run 7 | 0.78 | 0.82 | 0.82 | 0.83 | 0.84 |
| Run 8 | 0.92 | 0.97 | 0.97 | 0.98 | 0.99 |
| Run 9 | 1.04 | 1.06 | 1.06 | 1.05 | 1.07 |
| Run 10 | 0.83 | 0.85 | 0.85 | 0.85 | 0.86 |
| Run 11 | 0.73 | 0.73 | 0.73 | 0.75 | 0.74 |
| Run 12 | 0.96 | 0.96 | 0.95 | 0.98 | 0.99 |
| Run 13 | 0.96 | 0.96 | 0.95 | 0.98 | 0.99 |
| ddPCR | | | | | |
| Run 1 | 0.94 | 0.92 | 1.03 | 1.03 | 0.87 |
| Run 2 | 0.96 | 0.99 | 0.95 | 0.97 | 1.05 |
| Run 3 | 0.98 | 0.93 | 0.92 | 0.84 | 0.95 |
| Run 4 | 1.10 | 1.11 | 1.08 | 1.06 | 0.96 |
| Run 5 | 1.06 | 1.07 | 1.06 | 1.13 | 1.12 |
| Run 6 | 0.96 | 0.99 | 0.95 | 0.97 | 1.05 |

Comparison of qPCR and ddPCR as Basis for Potency Assay

To assess the effects of vector genome titer variations on AAV vector biopotency assays, titers determined in the qPCR (using the procedures of Example 4) vs ddPCR (using the procedures of Example 5) were used as the starting point.

Biopotency assays was performed 5 times by 2 operators, with 3 replicate dilution series per run. Multiplicity of infection (MOI) was calculated from either the qPCR or ddPCR mean vector genome titer. The resulting Potency Inter-Assay Variability data is shown in Tables 5 and 6.

TABLE 5
Potency Inter-Assay Variability - qPCR

| | Batch 1 | Batch 2 | Batch 3 | Batch 4 | Reference |
|---|---|---|---|---|---|
| EC50 | | | | | |
| Run 1 | 3689 | 4781 | 6569 | 4841 | 4920 |
| Run 2 | 1979 | 2370 | 2238 | 2051 | 2086 |
| Run 3 | 1456 | 2722 | 2873 | 2712 | 2281 |
| Run 4 | 1568 | 2472 | 2748 | 2267 | 2332 |
| Run 5 | 3078 | 4378 | 4401 | 4038 | 3672 |
| Avarage | 2354 | 3345 | 3766 | 3182 | 3058 |
| Standard | 984 | 1143 | 1763 | 1206 | 1217 |
| Deviation | | | | | |
| % CV | 42 | 34 | 47 | 38 | 40 |
| Relative | | | | | |
| Potency % | | | | | |
| Run 1 | 133% | 103% | 75% | 102% | 133% |
| Run 2 | 105% | 88% | 93% | 102% | 105% |
| Run 3 | 157% | 84% | 79% | 84% | 157% |
| Run 4 | 149% | 94% | 85% | 103% | 149% |
| Run 5 | 119% | 84% | 83% | 91% | 119% |
| Avarage | 133% | 91% | 83% | 96% | 133% |
| Standard | 21% | 8% | 7% | 8% | 21% |
| Deviation | | | | | |
| % CV | 16% | 9% | 8% | 9% | 16% |

TABLE 6
Potency Inter-Assay Variability - ddPCR

| | Batch 1 | Batch 2 | Batch 3 | Batch 4 | Reference |
|---|---|---|---|---|---|
| EC50 | | | | | |
| Run 1 | 3736 | 5074 | 6994 | 5066 | 5322 |
| Run 2 | 2004 | 2516 | 2383 | 2147 | 2256 |
| Run 3 | 1475 | 2890 | 3060 | 2838 | 2467 |
| Run 4 | 1588 | 2624 | 2927 | 2372 | 2523 |
| Run 5 | 3117 | 4647 | 4687 | 4226 | 3972 |
| Avarage | 2384 | 3550 | 4010 | 3330 | 3308 |
| Standard | 996 | 1213 | 1877 | 1263 | 1316 |
| Deviation | | | | | |
| % CV | 42 | 34 | 47 | 38 | 40 |
| Relative | | | | | |
| Potency % | | | | | |
| Run 1 | 142% | 105% | 76% | 105% | 142% |
| Run 2 | 113% | 90% | 95% | 105% | 113% |
| Run 3 | 167% | 85% | 81% | 87% | 167% |
| Run 4 | 159% | 96% | 86% | 106% | 159% |
| Run 5 | 127% | 85% | 85% | 94% | 127% |
| Avarage | 142% | 92% | 84% | 99% | 142% |
| Standard | 22% | 8% | 7% | 9% | 22% |
| Deviation | | | | | |
| % CV | 16% | 9% | 8% | 9% | 16% |

Mean vector genome titers from qPCR and ddPCR were not statistically different, showing that the ddPCR method is equivalent to qPCR for vector genome titering

Mean titers from qPCR and ddPCR, when used as input for a cell-based biopotency assay, yielded almost identical EC50's and relative potencies, again showing ddPCR is a valid method to use for vector genome titering.

The use of a relative potency readout (%reference) vs an absolute potency readout (EC50) reduces the inter-assay variability of the biopotency assay by more than two-fold from 34-42% for the absolute readout to 8-16% for the relative readout.

What is claimed:

1. A method for measuring the potency of AAV vector particles in a first formulation, comprising:
providing a first formulation comprising a first collection of AAV vector particles, wherein the first collection of AAV vector particles comprise a polyncleotide encoding a payload molecule;
determing the titer of the AAV vector particles in the first formulation using qPCR, ddPCR or a combination thereof; and
measuring the potency of the AAV vector particles from the first formulation by:
determining a multiplicity of infection (MOI) for the first collection of AAV vector particles based on the titer of the AAV vector particles in the first formulation;
transducing the AAV vector particles from the first formulation into a target cell using the MOI for the first collection of AAV vector particles, and under conditions in which the target cell will produce the payload molecule; and
measuring the amount of payload molecule produced from the AAV vector particles, such that the potency of the AAV vector particle is measured.

2. The method of claim 1, wherein the titer of the AAV vector particles in the first formulation is determined using qPCR.

3. The method of claim 1, wherein the titer of the AAV vector particles in the first formulation is determined using ddPCR.

4. The method of any one of claims 1-3, wherein the step of measuring the amount of payload molecule produced from the first collection of AAV vector particles comprises:
lysing the target cells and collecting the resulting cell lysate sample; adding a molecule of interest to the cell lysate sample, wherein the molecule of interest interacts with the payload molecule to produce a product molecule; and measuring the amount of product molecule produced in the cell lysate, such that the potency of the AAV vector particles from the first formulation is measured.

5. The method of claim 4, wherein the amount of product molecule produced is measured using Ultra High-Pressure Liquid Chromatography (UHPLC).

6. The method of any one of claims 1-5, wherein the method further comprises: comparing the potency of AAV vector particles in the first formulation to the potency of reference AAV vector particles in a viral vector reference standard.

7. The method of claim 6, wherein the potency of the AAV vector particles in the viral vector reference standard is measured according to the following steps:
providing a reference formulation comprising a collection of reference AAV vector particles, wherein the collection of reference AAV vector particles comprise a polyncleotide encoding the payload molecule;
determing the titer of the reference AAV vector particles in the reference formulation using qPCR, ddPCR or a combination thereof;
measuring the potency of the reference AAV vector particles from the reference formulation by:
determining a multiplicity of infection (MOI) for the reference collection of AAV vector particles based on the titer of the reference AAV vector particles in the reference formulation;
transducing the reference AAV vector particles from the reference formulation into a target cell using the MOI for the reference collection of AAV vector particles, and under conditions in which the target cell will produce the payload molecule; and
measuring the amount of payload molecule produced from the reference AAV vector particles, such that the potency of the reference AAV vector particle is measured.

8. The method of claim 7, wherein the titer of the reference AAV vector particles in the reference formulation is determined using qPCR.

9. The method of claim 7, wherein the titer of the reference AAV vector particles in the reference formulation is determined using ddPCR.

10. The method of claim 7, wherein the titer of the AAV vector particles in the first formulation is determined using qPCR; and wherein the titer of the reference AAV vector particles in the reference formulation is determined using ddPCR.

11. The method of claim 7, wherein the titer of the AAV vector particles in the first formulation is determined using ddPCR; and wherein the titer of the reference AAV vector particles in the reference formulation is determined using qPCR.

12. The method of any one of claims 1-11, wherein the target cells are HT1080 cells.

13. The method of claim 12, wherein the HT1080 cells are plated onto a testing plate at a density of 1×10⁴ cells/well.

14. A method for measuring the titer of AAV vector particles in a formulation, comprising: providing a formulation comprising a collection of AAV vector particles; and determing the titer of the AAV vector particles in the formulation using ddPCR.
